(19) United States
(12) Patent Application Publication
(10) Pub. No.: US 2009/0041673 A1
(43) Pub. Date: Feb. 12, 2009

(54) Thermally Crosslinked Contrast Agents

(75) Inventors: Sangyong Jon, Daejeon (KR); Yong Yeon Jeong, Gwangju (KR); Haerim Lee, Ulsan (KR); Mi Kyung Yu, Seoul (KR); Sangjin Park, Suwon-si (KR); Jae II Kim, Gwangju (KR)

Correspondence Address: SUGHRUE MION, PLLC, 2100 PENNSYLVANIA AVENUE, N.W., SUITE 800, WASHINGTON, DC, 20037, US

(73) Assignee: Anygen Co., Ltd., Gwangju (KR)

(21) Appl. No.: 11/833,873

(22) Filed: Aug. 3, 2007

(51) Int. Cl. A61K 49/18; A61K 49/04

(52) U.S. Cl. 424/9.322; 424/9.4

(57) ABSTRACT

The present invention relates to a contrast agent comprising a plurality of nanoparticles, wherein each of the nanoparticles comprises: (a) a signal generating core; and (b) a polymeric shell coated on the signal generating core, wherein the polymeric shell comprises a water soluble hydoxysilyl- or alkoxysilyl-functionalized polymer and wherein the polymer shell is thermally crosslinked through hydroxysilyl or alkoxysilyl groups of the polymer.

### BACKGROUND OF THE INVENTION

1. Field of the Invention

The present invention relates to a contrast agent comprising a plurality of nanoparticles, a process for preparing a contrast agent comprising a plurality of nanoparticles and a method for providing an image of an internal region of a patient.

2. Description of the Related Art

Superparamagnetic iron oxide nanoparticles (SPION) are an emerging form of nanomedicine¹ for the treatment of various diseases including cancer because they can be used as magnetic resonance (MR) contrast agents²⁻¹⁰ and can be incorporated into magnetic field-guided drug delivery vehicles for cancer treatment¹²⁻¹⁴. Several requirements must be met for SPION to be successfully used in MR imaging in vivo: (i) good dispersibility in physiological medium, (ii) biocompatibility, and (iii) an anti-biofouling property that prevents adsorption of plasma proteins or cells onto their surface. Therefore, it is necessary to engineer the surface of SPION to minimize such biofouling and aggregation under harsh physiological conditions (i.e., high salt and protein concentrations).¹⁵⁻²² Several synthetic and natural polymers have been employed to coat the surface of SPION.^{15,16,21,22} These polymers include dextran,^{23,24} polyethylene glycols (PEGs),^{12,15,16,25} and polyvinylpyrrolidone,^{16,26} all of which are known to be biocompatible and all of which promote good dispersion of SPION in aqueous medium; however, the possibility that the polymer coating can be lost under harsh in vivo conditions has been a concern.^{16,21,22}

To ensure the stability of the polymer coating in vivo, crosslinked iron oxide nanoparticles (CLIO) had been developed.^{23,27} CLIO are composed of dextran-coated SPION in which the dextran polymer chains are chemically crosslinked. These nanoparticles have been widely used for in vivo as well as in vitro MR imaging.²⁸⁻³¹ Although CLIO are considered one of the most promising MR contrast agents because of their stable coating, antibiofouling property, biocompatibility, and small hydrodynamic size, multiple synthetic and purification steps are needed to obtain the final, functionalized SPION, namely, (i) dextran coating and purification; (ii) crosslinking using epichlorohydrin and purification; and (iii) chemical treatment to introduce functional groups such as amines and aldehydes onto the nanoparticle surface.²⁹ Furthermore, there is a concern that the unreacted residual chemical reagents in CLIO will be toxic in vivo.

Throughout this application, various patents and publications are referenced and citations are provided in parentheses. The disclosure of these patents and publications in their entities are hereby incorporated by references into this application in order to more fully describe this invention and the state of the art to which this invention pertains.

### SUMMARY OF THE INVENTION

The present inventors have made intensive researches to develop contrast agents showing excellent performance in imaging technologies. As a result, the present inventors have found that the thermal crosslinking shell of water soluble hydoxysilyl- or alkoxysilyl-functionalized polymers on signal generating cores allows contrast agents to show improved properties such as higher stability in physiological medium, resistance to uptake by cells in the reticuloendothelial system, enhanced permeability and retention effect.

Accordingly, it is an object of this invention to provide a contrast agent comprising a plurality of nanoparticles.

It is another object of this invention to provide a process for preparing a contrast agent comprising a plurality of nanoparticles.

It is further object of this invention to provide a method for providing an image of an internal region of a patient.

Other objects and advantages of the present invention will become apparent from the following detailed description together with the appended claims and drawings.

### BRIEF DESCRIPTION OF THE DRAWINGS

FIG. 1 schematically represents the TCL-SPION (thermally crosslinked superparamagnetic iron oxide nanoparticles) of the present invention.

FIG. 2 schematically illustrates the carboxyl TCL-SPION showing crosslinking between polymer layers after heat treatment.

FIG. 3 is a hydrodynamic size distribution graph along with TEM image of the carboxyl TCL-SPION. The scale bar in the TEM image corresponds to 10 nm.

FIG. 4 is a FT-IR spectra of the carboxyl TCL-SPION.

FIG. 5 is a TGA graph of the carboxyl TCL-SPION.

FIG. 6 represents variation of the magnetization of the carboxyl TCL-SPION as a function of applied magnetic field.

FIG. 7 shows high-resolution Si(2p) XPS data for the carboxyl TCL-SPION before (a) and after (b) heat treatment at 80° C. for 2 hr.

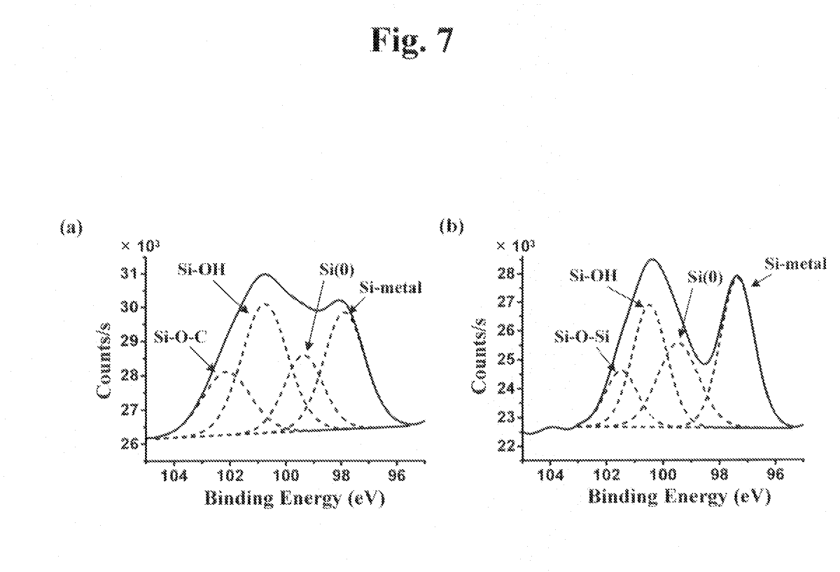

FIG. 8 represents synthetic scheme for the amino and Cy5.5 TCL-SPION.

FIG. 9 is a fluorescence spectrum of the Cy5.5 TCL-SPION.

FIG. 10 is photographs of the carboxyl TCL-SPION and amino TCL-SPION dispersed in phosphate-buffered saline (pH 7.4).

FIG. 11A represents (a) T2-weighted fast-spin echo images (TR/TE of 4200 ms/102 ms) taken at 0 hr and 3.5 hr post-injection of 14.7 mg Fe/kg of the Cy5.5 TCL-SPION at the level of tumor (320 mm³) on the flank above the upper left thigh of a nude mouse. The dashed circle with the arrow indicates the allograft tumor region; and (b) Optical fluorescence images of the same mouse taken at 0 hr and 3.5 hr post-injection. Images were acquired with an exposure time of 1 sec and with the Cy5.5 filter channel. The red arrows indicate the position of the allograft tumor. The colored scale indicates the relative fluorescence intensity.

FIG. 11B represents T2-weighted fast-spin echo images (TR/TE of 4200 ms/102 ms) taken at 0 hr (a) and 1 hr post-injection of 10 mg Fe/kg of the silicon-grafted chitosan coated TCL-SPION at the level of the tumor on the back of a nude mouse.

FIG. 11C represents T2-weighted fast-spin echo images (TR/TE of 4200 ms/102 ms) taken at 0 hr (a) and 1 hr post-injection of 10 mg Fe/kg of the silicon-grafted carboxymethyl dextran coated TCL-SPION at the level of the tumor on the back of a nude mouse.

FIG. 12 represents photographic images (a) and corresponding optical fluorescence images (b) of several organs and the allograft tumor. The images were taken after sacrifice (at 3.5 hr post injection) of the same mouse as used in the MR/optical imaging experiment (injected with 14.7 mg Fe/kg of Cy5.5 TCL-SPION). Fluorescence images were acquired with an exposure time of 1 sec and using the Cy5.5 filter channel. The colored scale indicates the relative fluorescence intensity.

### DETAILED DESCRIPTION OF THIS INVENTION

In one aspect of this invention, there is provided a contrast agent comprising a plurality of nanoparticles, wherein each of the nanoparticles comprises:
(a) a signal generating core; and
(b) a polymeric shell coated on the signal generating core, wherein the polymeric shell comprises a water soluble hydoxysilyl- or alkoxysilyl-functionalized polymer and wherein the polymer shell is thermally crosslinked through hydroxysilyl or alkoxysilyl groups of the polymer.

The present inventors have made intensive researches to develop contrast agents showing excellent performance in imaging technologies. As a result, the present inventors have found that the thermal crosslinking shell of water soluble hydoxysilyl- or alkoxysilyl-functionalized polymers on signal generating cores allows contrast agents to show improved properties such as higher stability in physiological medium, resistance to uptake by cells in the reticuloendothelial system, enhanced permeability and retention effect.

As used herein, the term “hydoxysilyl-functionalized polymer” means a polymer comprising hydroxysilyl moieties as side chains. The term “hydroxysilyl” means hydroxyl-substituted silyl groups, preferably, trihydroxysilyl.

As used herein, the term “alkoxysilyl-functionalized polymer” means a polymer comprising alkoxysilyl moieties as side chains. The term “alkoxysilyl” means alkoxy-substituted silyl groups, preferably trimethoxysilyl and triethoxysilyl, more preferably, trimethoxysilyl.

For nanoparticles of this invention, a variety of signal generating cores may be used. Preferably, the signal generating core is a paramagnetic, superparamagnetic, proton density or X-ray active (Computed Tomography) signal generating core, more preferably, superparamagnetic signal generating core containing an iron oxide.

Exemplary paramagnetic signal generating cores suitable for use in the present invention include stable free radicals (such as stable nitroxides), as well as compounds comprising transition, lanthanide and actinide elements. Preferable elements include Gd(III), Mn(II), Cu(II), Cr(III), Fe(II), Fe(III), Co(II), Er(II), Ni(II), Eu(III) and Dy(III).

Exemplary superparamagnetic signal generating cores suitable for use in the present invention include ferro- or ferrimagnetic compounds, such as pure iron, magnetic iron oxide (such as magnetite, Fe₃O₄), γ-Fe₂O₃, manganese ferrite, cobalt ferrite and nickel ferrite. Preferably, superparamagnetic signal generating cores suitable for use in the present invention include Fe₃O₄ and γ-Fe₂O₃, more preferably, Fe₃O₄.

Exemplary proton density signal generating cores include perfluorocarbons.

X-ray active signal generating cores suitable for use in the present invention include iodine, bismuth sulfide, and gold. Preferably, X-ray active signal generation core suitable for use in the present invention includes gold.

The signal generating core is coated with a water soluble polymeric shell. The thermally-crosslinked polymer shell permits the contrast agent of this invention to show water solubility and anti-biofouling properties. Also, the polymeric shell is responsible for improvement in stability and imaging capacity of the contrast agent.

The shell is composed of silicon-grafted polymers with water solubility. The main backbones of the silicon-grafted polymers comprise synthetic polymers and natural polymers.

According to a preferred embodiment, the synthetic polymer as main backbones for the silicon-grafted polymers is selected from the groups consisting of poly(acrylic acid) and its derivates, poly(methacrylic acid) and its derivates [e.g., poly(methylmethacrylic acid), poly(ethylmethacrylic acid), poly(butylmethacrylic acid), poly(laurylmethacrylic acid), poly(hydroxyethylmethacrylic acid) and poly(hydroxypropylmethacrylic acid)], poly(acrylamide) and its derivates, poly(isocyanate) and its derivates, poly(styrene) and its derivates, poly(ethylene imine) and its derivates [e.g., PEGylated poly(ethylene imine)], poly(siloxane) and its derivates, poly(glutamic acid) and its derivates [e.g., PEGylated poly(glutamic acid)], poly(aspartic acid) and its derivates [e.g., PEGylated poly(aspartic acid)], poly(lysine) and its derivates [e.g., PEGylated poly(lysine)], poly(arginine) and its derivates [e.g., PEGylated poly(lysine)], polypropylene glycol, poly(vinyl alcohol) and its derivates, poly(vinyl pyrrolidone) and its derivates, and polyethylene oxide (PEG) and its derivates.

More preferably, the synthetic polymer as main backbones is poly(acrylic acid) or poly(methacrylic acid).

According to a preferred embodiment, the natural polymer as main backbones for the silicon-grafted polymers is selected from the group consisting of chitosan and its derivatives, dextran and its derivatives (e.g., carboxymethyl dextran), cellulose and its derivatives (e.g., carboxymethyl cellulose), heparin and its derivatives, hyaluronic acid and its derivatives, and alginate and its derivatives.

The silicon atoms contained the water soluble polymer may be grafted to the main backbones in various manners. For example, the silicon atoms may be grafted to the main backbones by monoester linkages. Where the silicon atoms are grafted to the main backbones by monoester linkages, they may be directly linked or indirectly linked to the main backbones through a C₁-C₅ hydrocarbon moiety.

According to a preferred embodiment, silicon atoms contained in the water soluble polymer is covalently linked to the signal generating core. The covalent linkage increases stability of the polymeric shell coating on the signal generating core.

It is one of the most prominent features of the present invention to utilize a thermally-crosslinked polymer shell coated on signal generating cores. The crosslinking of the polymeric shell can be achieved by simple heating treatments (e.g., heat treatment at 70-80° C.). The crosslinking contributes particularly to improvement in stability of the polymeric shell on the signal generating core. Also, the thermally-crosslinked polymer shell on the signal generating core gives rise to production of more favorable imaging results than non-crosslinked polymer shells.

According to a preferred embodiment, the water soluble polymer is further functionalized with polyethylene glycol (PEG). PEG showing water solubility has an antibiofouling property to prevent nonspecific adsorption of plasma proteins and cells onto nanoparticles. PEG may be grafted to the main backbones in various manners. For example, PEG may be grafted to the main backbones by monoester linkages. Where the silicon atoms are grafted to the main backbones by monoester linkages, they may be directly linked or indirectly linked to the main backbones through a C₁-C₅ hydrocarbon moiety. The water soluble polymer functionalized with PEG permits nanoparticles to exhibit enhanced water dispersibility.

The most striking feature of the present invention is a peculiar silicon atomic composition of the thermally crosslinked polymeric shell.

According to a preferred embodiment, the thermally crosslinked polymeric shell formed by the silicon-containing water soluble polymer on the signal generating core has the relative silicon atomic composition of 5-50% in the form of Si—O—Si of all types of Si bonds in the polymeric shell. The silicon atoms in the form of Si—O—Si bonds are involved in thermal crosslinking of polymeric shells. More preferably, the thermally crosslinked polymeric shell formed by the silicon-containing water soluble polymer on the signal generating core has the relative silicon atomic composition of 10-30% (still more preferably, 10-20%, most preferably, 10-15%) in the form of Si—O—Si of all types of Si bonds in the polymeric shell.

According to a preferred embodiment, the thermally crosslinked polymeric shell formed by the silicon-containing water soluble polymer on the signal generating core has the relative silicon atomic composition of 10-30% in the form of Si—O—Si, 10-40% in the form of Si—OH, and 0-5% in the form of Si—O—C of all types of Si bonds in the polymeric shell. The form of Si—OH may be generated from hydrolysis of alkoxysilyl groups in the water soluble polymer. The form of Si—O—C may be derived from alkoxysilyl groups in the water soluble polymer. More preferably, the thermally crosslinked polymeric shell formed by the silicon-containing water soluble polymer on the signal generating core has the relative silicon atomic composition of 10-20% (most preferably, 10-15%) in the form of Si—O—Si, 18-34% (most preferably, 25-32%) in the form of Si—OH, and 0% in the form of Si—O—C of all types of Si bonds in the polymeric shell.

The advantages and favorable results of the present invention are ascribed greatly to the relative silicon atomic composition described above.

According to a preferred embodiment, the polymeric shell further comprises an antibody, peptide, nucleic acid (DNA or RNA) or dye. The conjugated antibodies, peptides and nucleic acid molecules intend to permit nanoparticles to be specifically bound to target biomolecules, cells or tissues. The dye molecules are utilized for optical imaging. The targeting ligands or dyes may be directly or indirectly linked to the polymer through linkers or spacers.

Where polymeric shell further comprises dye molecules (in particular, fluorescent dyes), the contrast agent of this invention provides a dual imaging probe (magnetic resonance/optical).

Dyes useful in this invention include, but not limited to, pyrene, an anthracene, a naphthalene, an acridine, a stilbene, an indole, an oxazole, benzoxazole, a thiazole, a benzothiazole, a 4-amino-7-nitrobenz-2-oxa-1,3-diazole (NBD), a carbocyanine, a carbostyryl, a porphyrin, a salicylate, an anthranilate, an azulene, a perylene, a pyridine, a quinoline, a xanthene, an oxazine, a benzoxazine, a carbazine, a phenalenone, a coumarin, a benzofuran, a benzphenalenone, a semiconductor nanocrystal and a fluorescent protein. In particular, fluorescent dyes useful in this invention include indocyanine green, cyanine 5.5, cyanine 7.5, fluorescein, rhodamine, yellow fluorescent protein, green fluorescent protein, fluorescein isothiocyanate and derivatives thereof, but not limited to.

Surprisingly, the present contrast agent comprising a fluorescent dye enables to produce a dual image for tissues (preferably, cancer tissues). As demonstrated in Examples, the present contrast agent comprising a fluorescent dye appears to be the first successful use of a SPION system for MR/optical dual imaging of cancer in vivo.

According to a preferred embodiment, the water soluble polymer prior to thermal crosslinking is represented by the following formula I:

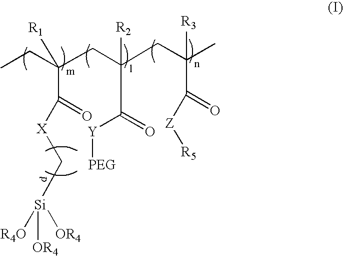

wherein R₁, R₂ and R₃ independently represent H or C₁-C₅ alkyl; R₄ represents H or C₁-C₅ alkyl; R₅ represents H, C₁-C₅ alkyl or heteroalkyl, (alkoxysilyl)alkyl, (hydroxysilyl)alkyl, 5-7 membered monocylic heterocycle consisting of carbon atoms and 1-3 heteroatoms, antibody, peptide, nucleic acid or dye; X, Y and Z independently represent oxygen or nitrogen atom; PEG represents polyethylene glycol; m, l and n independently represent an integer of 1-1,000; and p represents an integer of 1-10.

The polymer represented by the formula I may be block copolymers or random copolymers, preferably, random copolymers.

As used herein, the term “alkyl” means a branched or unbranched saturated or unsaturated hydrocarbon chain comprising a designated number of carbon atoms. For example, C₁-C₅ straight or branched alkyl hydrocarbon chain contains 1 to 5 carbon atoms, and includes but is not limited to methyl, ethyl, propyl, iso-propyl, butyl, iso-butyl, tert-butyl, n-pentyl and the like, unless otherwise indicated.

The term “heteroalkyl” means a branched or unbranched saturated or unsaturated hydrocarbon chain with the suitable replacement of carbon atoms with some other atom such as nitrogen, oxygen and sulfur which would render a chemically stable species.

The term “(alkoxysilyl)alkyl” refers to alkoxy-substituted silylalkyl groups. For example, the term (alkoxysilyl)alkyl includes trimethoxysilylethyl, trimethoxysilylpropyl, trimethoxysilylbutyl, trimethoxysilylpentyl, triethoxysilylethyl, triethoxysilylpropyl, triethoxysilylbutyl, methyldimethoxysilylethyl, methyldimethoxysilylpropyl, dimethylmethoxysilylethyl and dimethylmethoxysilylpropyl.

The term “(hydroxysilyl)alkyl” refers to hydroxyl-substituted silylalkyl groups, e.g., including trihydroxysilylethyl, trihydroxysilylpropyl, trihydroxysilylbutyl and trihydroxysilylpentyl.

The term “heterocycle” used herein is intended to mean 5-7 membered monocylic heterocylic ring which is saturated, partially unsaturated or unsaturated (aromatic), and which consists of carbon atoms and 1-3 heteroatoms independently selected from the group consisting of N, O and S. Preferably, the heterocyle includes succinimide, sulfosuccinimide, and maleimide.

In the formula I, it is preferred that R₁, R₂ and R₃ independently represent H or methyl. It is preferred that R₄ represents H or methyl, more preferably, H.

Preferably, R₅ represents H, C₁-C₅ alkyl, (alkoxysilyl)alkyl, (hydroxysilyl)alkyl, antibody or dye. Where nanoparticles of this invention is used for only MR imaging, it is preferred that R₅ represents (alkoxysilyl)alkyl or (hydroxysilyl)alkyl, more preferably, (trimethoxysilyl)alkyl, (triethoxysilyl)alkyl or (trihydoxysilyl)alkyl, still more preferably, (trimethoxysilyl)ethyl, (trimethoxysilyl)propyl, (trimethoxysilyl)butyl, (trimethoxysilyl)pentyl, (triethoxysilyl)ethyl, (triethoxysilyl)propyl, (triethoxysilyl)butyl, (triethoxysilyl)pentyl, (trihydoxysilyl)ethyl, (trihydoxysilyl)propyl, (trihydoxysilyl)butyl or (trihydoxysilyl)pentyl, still yet more preferably, (trimethoxysilyl)ethyl, (trimethoxysilyl)propyl, (trimethoxysilyl)butyl, (trimethoxysilyl)pentyl, (trihydoxysilyl)ethyl, (trihydoxysilyl)propyl, (trihydoxysilyl)butyl or (trihydoxysilyl)pentyl, and most preferably, (trihydoxysilyl)propyl.

Where nanoparticles of this invention are used for dual imaging (MR/optical), it is preferred that R₅ represents fluorescent dyes.

In the formula I, it is preferred that X, Y and Z independently represent an oxygen atom. More preferably, all of X, Y and Z represent oxygen atoms. Preferably, p is an integer of 1-5, more preferably, 2-4.

According to a preferred embodiment, the water soluble polymer prior to thermal crosslinking comprises poly(aspartic acid) as main backbones represented by the following formula II:

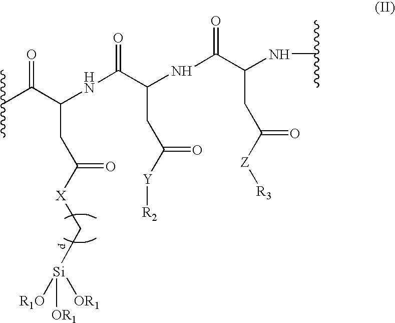

wherein R₁ represents H or C₁-C₅ alkyl; R₂ and R₃ independently represent H, C₁-C₅ alkyl or heteroalkyl, (alkoxysilyl)alkyl, (hydroxysilyl)alkyl, polyethylene glycol, antibody, peptide, nucleic acid or dye; X, Y and Z independently represent oxygen or nitrogen atom; and p represents an integer of 1-10.

In the formula II, it is preferred that R₁ represents H or methyl, more preferably, H.

Preferably, R₂ and R₃ independently represent H, C₁-C₅ alkyl, (alkoxysilyl)alkyl, (hydroxysilyl)alkyl, polyethylene glycol, antibody or dye. Where nanoparticles of this invention is used for only MR imaging, it is preferred that one of R₂ and R₃ is PEG and the other is (alkoxysilyl)alkyl or (hydroxysilyl)alkyl, more preferably, (trimethoxysilyl)alkyl, (triethoxysilyl)alkyl or (trihydoxysilyl)alkyl, still more preferably, (trimethoxysilyl)ethyl, (trimethoxysilyl)propyl, (trimethoxysilyl)butyl, (trimethoxysilyl)pentyl, (triethoxysilyl)ethyl, (triethoxysilyl)propyl, (triethoxysilyl)butyl, (triethoxysilyl)pentyl, (trihydoxysilyl)ethyl, (trihydoxysilyl)propyl, (trihydoxysilyl)butyl or (trihydoxysilyl)pentyl, still yet more preferably, (trimethoxysilyl)ethyl, (trimethoxysilyl)propyl, (trimethoxysilyl)butyl, (trimethoxysilyl)pentyl, (trihydoxysilyl)ethyl, (trihydoxysilyl)propyl, (trihydoxysilyl)butyl or (trihydoxysilyl)pentyl, and most preferably, (trihydoxysilyl)propyl.

Where nanoparticles of this invention are used for dual imaging (MR/optical), it is preferred that one of R₂ and R₃ represents fluorescent dyes.

In the formula II, it is preferred that X, Y and Z independently represent an oxygen atom. More preferably, all of X, Y and Z represent oxygen atoms. Preferably, p is an integer of 1-5, more preferably, 2-4.

According to a preferred embodiment, the water soluble polymer prior to thermal crosslinking comprises chitosan as main backbones represented by the following formula III:

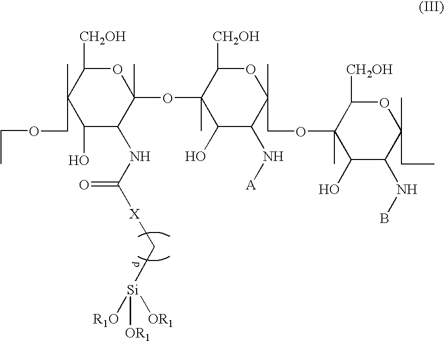

wherein R₁ represents H or C₁-C₅ alkyl; A and B independently represent H or —C(O)—Y—R₂; at least one of A and B is H; R₂ represents H, C₁-C₅ alkyl or heteroalkyl, (alkoxysilyl)alkyl, (hydroxysilyl)alkyl, polyethylene glycol, antibody, peptide, nucleic acid or dye; X and Y independently represent oxygen or nitrogen atom; and p represents an integer of 1-10.

In the formula II, it is preferred that R₁ represents H or methyl.

A and B independently represent H or —C(O)—Y—R₂; at least one of A and B is H. Preferably, R₂ represents H, C₁-C₅ alkyl or heteroalkyl, (alkoxysilyl)alkyl, (hydroxysilyl)alkyl, polyethylene glycol (PEG), antibody or dye.

Where nanoparticles of this invention are used for only MR imaging, it is preferred that R₂ represents PEG, (alkoxysilyl)alkyl or (hydroxysilyl)alkyl. More preferably, R₂ represents PEG.

Where R₂ is (alkoxysilyl)alkyl or (hydroxysilyl)alkyl, it represents preferably (trimethoxysilyl)alkyl, (triethoxysilyl)alkyl or (trihydoxysilyl)alkyl, still more preferably, (trimethoxysilyl)ethyl, (trimethoxysilyl)propyl, (trimethoxysilyl)butyl, (trimethoxysilyl)pentyl, (triethoxysilyl)ethyl, (triethoxysilyl)propyl, (triethoxysilyl)butyl, (triethoxysilyl)pentyl, (trihydoxysilyl)ethyl, (trihydoxysilyl)propyl, (trihydoxysilyl)butyl or (trihydoxysilyl)pentyl, still yet more preferably, (trimethoxysilyl)ethyl, (trimethoxysilyl)propyl, (trimethoxysilyl)butyl, (trimethoxysilyl)pentyl, (trihydoxysilyl)ethyl, (trihydoxysilyl)propyl, (trihydoxysilyl)butyl or (trihydoxysilyl)pentyl, and most preferably, (trihydoxysilyl)propyl.

Where nanoparticles of this invention is used for dual imaging (MR/optical), it is preferred that R₂ represents fluorescent dyes.

In the formula II, it is preferred that X and Y independently represent an oxygen atom. More preferably, both of X and Y represent oxygen atoms. Preferably, p is an integer of 1-5, more preferably, 2-4.

According to a preferred embodiment, the water soluble polymer prior to thermal crosslinking comprises carboxymethyl dextran as main backbones represented by the following formula IV:

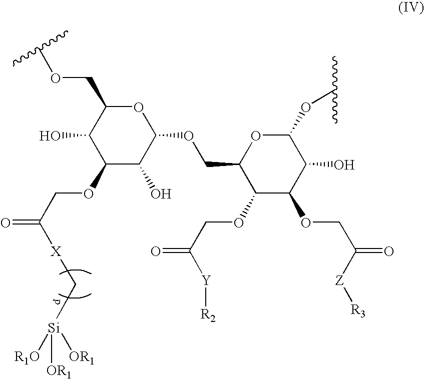

wherein R₁ represents H or C₁-C₅ alkyl; R₂ and R₃ independently represent H, C₁-C₅ alkyl or heteroalkyl, (alkoxysilyl)alkyl, (hydroxysilyl)alkyl, polyethylene glycol, antibody, peptide, nucleic acid or dye; X, Y and Z independently represent oxygen or nitrogen atom; and p represents an integer of 1-10.

In the formula IV, it is preferred that R₁ represents H or methyl, more preferably, H.

Preferably, R₂ and R₃ independently represent H, C₁-C₅ alkyl, (alkoxysilyl)alkyl, (hydroxysilyl)alkyl, polyethylene glycol, antibody or dye. Where nanoparticles of this invention is used for only MR imaging, it is preferred that one of R₂ and R₃ is PEG and the other is (alkoxysilyl)alkyl or (hydroxysilyl)alkyl, more preferably, (trimethoxysilyl)alkyl, (triethoxysilyl)alkyl or (trihydoxysilyl)alkyl, still more preferably, (trimethoxysilyl)ethyl, (trimethoxysilyl)propyl, (trimethoxysilyl)butyl, (trimethoxysilyl)pentyl, (triethoxysilyl)ethyl, (triethoxysilyl)propyl, (triethoxysilyl)butyl, (triethoxysilyl)pentyl, (trihydoxysilyl)ethyl, (trihydoxysilyl)propyl, (trihydoxysilyl)butyl or (trihydoxysilyl)pentyl, still yet more preferably, (trimethoxysilyl)ethyl, (trimethoxysilyl)propyl, (trimethoxysilyl)butyl, (trimethoxysilyl)pentyl, (trihydoxysilyl)ethyl, (trihydoxysilyl)propyl, (trihydoxysilyl)butyl or (trihydoxysilyl)pentyl, and most preferably, (trihydoxysilyl)propyl.

Where nanoparticles of this invention are used for dual imaging (MR/optical), it is preferred that one of R₂ and R₃ represents fluorescent dyes.

In the formula IV, it is preferred that X, Y and Z independently represent an oxygen atom. More preferably, all of X, Y and Z represent oxygen atoms. Preferably, p is an integer of 1-5, more preferably, 2-4.

According to a preferred embodiment, the water soluble polymer prior to thermal crosslinking comprises heparin as main backbones represented by the following formula V:

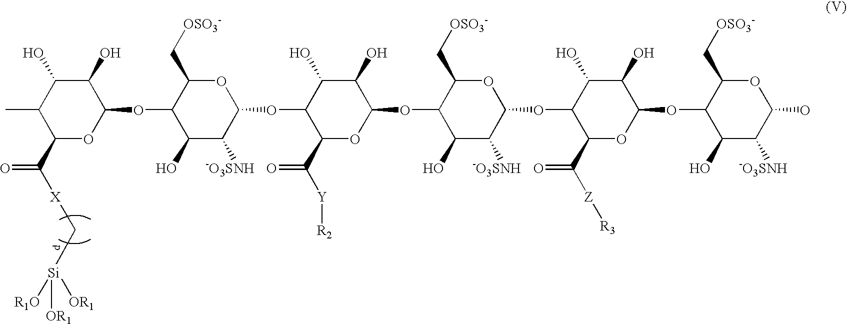

wherein R₁ represents H or C₁-C₅ alkyl; R₂ and R₃ independently represent H, C₁-C₅ alkyl or heteroalkyl, (alkoxysilyl)alkyl, (hydroxysilyl)alkyl, polyethylene glycol, antibody, peptide, nucleic acid or dye; X, Y and Z independently represent oxygen or nitrogen atom; and p represents an integer of 1-10.

In the formula V, it is preferred that R₁ represents H or methyl, more preferably, H.

Preferably, R₂ and R₃ independently represent H, C₁-C₅ alkyl, (alkoxysilyl)alkyl, (hydroxysilyl)alkyl, polyethylene glycol, antibody or dye. Where nanoparticles of this invention is used for only MR imaging, it is preferred that one of R₂ and R₃ is PEG and the other is (alkoxysilyl)alkyl or (hydroxysilyl)alkyl, more preferably, (trimethoxysilyl)alkyl, (triethoxysilyl)alkyl or (trihydoxysilyl)alkyl, still more preferably, (trimethoxysilyl)ethyl, (trimethoxysilyl)propyl, (trimethoxysilyl)butyl, (trimethoxysilyl)pentyl, (triethoxysilyl)ethyl, (triethoxysilyl)propyl, (triethoxysilyl)butyl, (triethoxysilyl)pentyl, (trihydoxysilyl)ethyl, (trihydoxysilyl)propyl, (trihydoxysilyl)butyl or (trihydoxysilyl)pentyl, still yet more preferably, (trimethoxysilyl)ethyl, (trimethoxysilyl)propyl, (trimethoxysilyl)butyl, (trimethoxysilyl)pentyl, (trihydoxysilyl)ethyl, (trihydoxysilyl)propyl, (trihydoxysilyl)butyl or (trihydoxysilyl)pentyl, and most preferably, (trihydoxysilyl)propyl.

Where nanoparticles of this invention are used for dual imaging (MR/optical), it is preferred that one of R₂ and R₃ represents fluorescent dyes.

In the formula V, it is preferred that X, Y and Z independently represent an oxygen atom. More preferably, all of X, Y and Z represent oxygen atoms. Preferably, p is an integer of 1-5, more preferably, 2-4.

According to a preferred embodiment, the contrast agent of this invention is used for magnetic resonance imaging (MRI). The contrast agent of this invention may be employed to provide MR image of various biological tissues. Preferably, the contrast agent exhibits considerable MR imaging capacity for cancer tissues.

To date, there have been only a few reports on direct imaging of cancers using polymer-coated SPION without attachment of specific ligands on their surfaces, presumably because such SPION lacking antibiofouling characteristics is easily taken up by the reticuloendothelial system. In contrast, since the contrast agent of this invention is fairly stable under physiological conditions and maintains its small size, it upon systemic circulation could be accumulated in tumor sites by the enhanced permeability and retention effect as a result of the presence of leaky vasculatures around tumors. Surprisingly, the contrast agent of this invention can successfully produce in vivo MR imaging of cancer tissues with no help of targeting ligands.

By “nanoparticles” is meant, particles having dimensions of from about 5 nanometers (nm) to 300 nm, more preferably from about 5 nm to about 100 nm, still more preferably from about 10 nm to about 70 nm, most preferably from about 20-50 nm. The sizes of the present nanoparticles are relatively smaller than those of the conventional dextran-coated SPION such as CLIO. In addition, the present nanoparticles show relatively narrow size distribution.

The smaller sizes and anti-biofouling coating of the present nanoparticles ensure them to easily penetrate into target tissues (e.g., cancer tissues), resulting in significant accumulation of nanoparticles into tissues. Such enhanced penetration potential is responsible partly for improvement in imaging capacity of the present nanoparticles. In addition, the smaller size and narrow size distribution of the thermally-crosslinked polymer coated nanoparticles contribute to the enhancement of magnetic signal intensity and homogeneity.

The nanoparticles of the present invention show enhanced saturation magnetization (Ms) values. Preferably, the nanoparticles exhibit superparamagnetic behaviors showing high Ms of from 10 to 200 emu/g Fe, more preferably, 30-150 emu/g Fe, still more preferably 50-100 emu/g Fe, most preferably 70-85 emu/g Fe. Considering the magnetic moment (30-50 emu/g Fe) of the conventional polymer-coated SPION, it could be recognized that the saturation magnetization values of the present nanoparticles is greatly improved. The higher Ms of the present nanoparticles is very advantageous in signal generation, resulting in increasing the imaging capacity.

The contrast agent of this invention shows significant anti-biofouling properties; moreover its thermally-crosslinked polymeric coating exhibits excellent stability under physiological conditions. In addition to these, the present contrast agent has a relatively small size. These characteristics described above enable the present contrast agent to be long maintained in the circulation system and to penetrate into tissues, resulting in the higher accumulation of the contrast agents in tissues of interest (e.g., cancer tissues). The present contrast agent is cleared from human body within a suitable period of time (e.g., one day). Based on the findings of this invention, it could be understood that the present contrast agent exhibits significantly improved imaging capacity through EPR effect and considerable safety to human. In particular, the present contrast agent shows a perfect performance as a cancer in vivo MRI probe.

In another aspect of this invention, there is provided a process for preparing a contrast agent comprising a plurality of nanoparticles, which comprises the steps of:
(a) forming a superparamagnetic signal generating core containing iron oxide by use of salts of Fe²⁺ and Fe³⁺;
(b) forming a water soluble polymeric shell on the signal generating core by adding a water soluble hydoxysilyl- or alkoxysilyl-functionalized polymer to the signal generating core and agitating; and
(c) heating the resultant of step (b) to thermally crosslink the polymeric shell coated on the signal generating core, whereby the contrast agent comprising a plurality of nanoparticles is prepared.

Alternatively, the present invention provides a process for preparing a contrast agent comprising a plurality of nanoparticles, which comprises the steps of:
(a) mixing salts of Fe²⁺ and Fe³⁺ and a water soluble hydoxysilyl- or alkoxysilyl-functionalized polymer to form nanoparticles comprising a superparamagnetic signal generating core and a water soluble polymeric shell;
(b) heating the resultant of step (a) to thermally crosslink the polymeric shell coated on the signal generating core, whereby the contrast agent comprising a plurality of nanoparticles is prepared.

Since the process of the present invention is intended to preparing the contrast agent of this invention described above, the common descriptions between them are omitted in order to avoid undue redundancy leading to the complexity of this specification.

According to the first process of this invention, it is first performed to form a superparamagnetic signal generating core containing iron oxide by use of salts of Fe²⁺ and Fe³⁺. Specifically, salts of Fe²⁺ and Fe³⁺0(e.g., FeCl₃ and FeCl₂) are dissolved in water, preferably, deoxygenated distilled water. To this solution is added an alkaline solution (e.g., aqueous ammonium solution) for elevating the pH to about 10 while stirring vigorously, resulting in the formation of iron oxide particles. The water soluble hydoxysilyl- or alkoxysilyl-functionalized polymer is added to iron oxide particles and agitated for coating. Then, the resultant is heat to thermally crosslink the polymeric shell coated on the signal generating core containing iron oxide particles. The heating treatment is preferably carried out at 50-80° C., more preferably 70-85° C.

The preparation process of this invention provides thermally crosslinked superparamagnetic iron oxide nanoparticles (TCL-SPION) with enhanced magnetic moment and decreased particle size.

In further aspect of this invention, there is provided a method for providing an image of an internal region of a patient, which comprises the steps of:
(a) administering to the patient a diagnostically effective amount of a contrast agent; wherein the contrast agent comprises a plurality of nanoparticles, wherein each of the nanoparticles comprises (i) a signal generating core containing an iron oxide; and (ii) a polymeric shell coated on the signal generating core, wherein the polymeric shell comprises a water soluble hydoxysilyl- or alkoxysilyl-functionalized polymer and wherein the polymer shell is thermally crosslinked through hydroxysilyl or alkoxysilyl groups of the polymer; and
(b) scanning the patient using a magnetic resonance imaging to obtain visible images of the region.

Since the method of the present invention uses the contrast agent of this invention described above, the common descriptions between them are omitted in order to avoid undue redundancy leading to the complexity of this specification.

The present method patient is useful in imaging an internal region of patients. The imaging process may be carried out by administering a diagnostically effective amount of the contrast agent to a patient, and then scanning the patient using magnetic resonance imaging to obtain visible images of an internal region (tissue) of a patient.

In particular, the present method is very advantageous for in vivo cancer imaging.

The contrast agent administered may be combined with a pharmaceutically acceptable carrier or vehicle. Pharmaceutically acceptable carriers and vehicles include, but are not limited to, carbohydrates (e.g., glucose, lactose, amylose, dextrose, sucrose, sorbitol, mannitol, starch, cellulose), gum acacia, calcium phosphate, alginate, gelatin, calcium silicate, microcrystalline cellulose, polyvinylpyrrolidone, water, salt solutions, alcohols, gum arabic, syrup, vegetable oils (e.g., corn oil, cotton-seed oil, peanut oil, olive oil, coconut oil), polyethylene glycols, methyl cellulose, methylhydroxy benzoate, propylhydroxy benzoate, talc, magnesium stearate and mineral oil.

The contrast agent may be administered orally or parenterally. For non-oral administration, intravenous injection, intramuscular injection, intra-articular, intra-synovial, intrasternal, intrathecal, intrahepatic, intralesional and intracranial injection may be employed.

The correct dosage of the contrast agent will be varied according to the particular formulation, the mode of application, age, body weight and sex of the patient, diet, time of administration, condition of the patient, reaction sensitivities and the like. It is understood that the ordinary skilled physician will readily be able to determine and prescribe a correct dosage of the contrast agent. The term used herein “diagnostically effective amount” refers to an amount suitable to provide MR images of patients.

The magnetic resonance imaging techniques which are employed are conventional and are described, for example, in D. M. Kean and M. A. Smith, Magnetic Resonance Imaging: Principles and Applications, (William and Wilkins, Baltimore 1986).

The features and advantages of the present invention will be summarized as follows:

(i) the present nanoparticles contained in contrast agents have a thermally crosslinked polymer shell coated on the signal generating core, wherein the polymeric shell a water soluble hydoxysilyl- or alkoxysilyl-functionalized polymer;

(ii) the thermal crosslinking through hydroxysilyl or alkoxysilyl groups of the polymer can be easily achieved by a heat treatment;

(iii) the nanoparticle of the present invention has an unique silicon atomic composition of the thermally crosslinked polymeric shell, which contributes greatly to the advantages and unexpected results of the present contrast agents;

(iv) the nanoparticle of the present invention has a relatively small size, which ensures nanoparticles to easily penetrate into target tissues (e.g., cancer tissues) and is responsible partly for improvement in imaging capacity of the present nanoparticles;

(v) the nanoparticles of the present invention show enhanced saturation magnetization (Ms) values, which results in increasing the imaging capacity;

(vi) the contrast agent of the present invention exhibits higher stability under physiological conditions, resistance to uptake by cells in the reticuloendothelial system, and enhanced permeability and retention effect;

(vii) the contrast agent of the present invention is very effective for tumor detection in vivo by MR or dual imaging even though it does not bear any targeting ligands;

(viii) such passive tumor targeting due to an enhanced permeability and retention effect could be achieved because of the antibiofouling and thermally-crosslinked stable polymer coating layers on the signal generating core; and

(ix) the present contrast agent comprising a fluorescent dye appears to be the first successful use of a SPION system for MR/optical dual imaging of cancer in vivo.

The present invention will now be described in further detail by examples. It would be obvious to those skilled in the art that these examples are intended to be more concretely illustrative and the scope of the present invention as set forth in the appended claims is not limited to or by the examples.

### EXAMPLES

### Example I

### Synthesis of Poly(TMSMA-r-PEGMA-r-NAS)³²

The synthesis of poly(TMSMA-r-PEGMA-r-NAS) was carried out according to Scheme I. 3-(trimethoxysilyl)propyl methacrylate (TMSMA, Sigma-Aldrich Chemical Co., 11.2 mmol, 2.78 g, 1 equiv), poly(ethylene glycol) methyl ether methacrylate (PEGMA, Sigma-Aldrich Chemical Co., 11.2 mmol, 5.32 g, 1 equiv), and N-acryloxysuccinimide (NAS, ACROS Organics, 9.6 mmol, 1.62 g, 0.86 equiv) were dissolved in 25 ml of tetrahydrofuran (THF, Sigma-Aldrich Chemical Co., anhydrous, 99.9%, inhibitor-free). This mixture was degassed for 15 min by bubbling with N₂. After adding 0.32 mmol of 2,2′-azobisisobutyronitrile (Sigma-Aldrich Chemical Co., 53 mg, 0.01 equiv) as a radical initiator, the vial was sealed with a Teflon-lined screw cap. The polymerization reaction was carried out at 70° C. for 24 hr. The final product solution was stored at 4° C. ¹H NMR (300.40 MHz, CDCl₃): δ=4.13 (br, 2H, CO₂—CH₂ of PEGMA), 3.92 (br, 2H, CO₂—CH₂ of TMSMA), 3.66 (s, 30H), 3.63-3.55 (s, 9H; m, 2H), 3.40 (s, 3H), 2.82 (br, 4H, CO—CH₂ of NHSA) 2.0-1.71 (br, 6H), 1.04 (br, 2H), 0.87 (br, 4H), 0.66 (br, 2H). Mₙ=16,274 and M_{w}=26,795 with a polydispersity of 1.646 as measured by gel permeation chromatography using a Waters 1515 isocratic pump and a Waters 2414 Refractive Index detector. THF was used as the eluent and delivered at a flow rate of 0.4 mL/min.

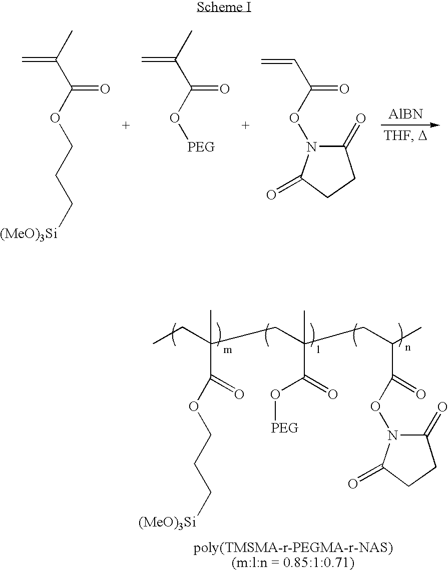

The final product poly(TMSMA-r-PEGMA-r-NAS) was hydrolyzed in water to give the hydrolyzed form represented by formula 1:

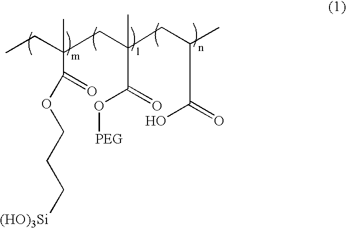

### EXAMPLE II

### Synthesis of Silicon-Grafted Poly(Aspartic Acid)

The synthesis of silicon-grafted poly(aspartic acid) was carried out according to Scheme II. 1.37 g (10 mmole of —COOH) of poly-(α,β)-_{DL}-aspartic acid sodium salt (2-10 kDa; Sigma-Aldrich Chemical Co) and 0.9 g (5 mmol) of 3-aminopropyltrimethoxysilane (Sigma-Aldrich Chemical Co) were dissolved in 20 mL distilled water. 7.6 g (40 mmol) of 1-Ethyl-3-(3-dimethylaminopropyl) carbodiimide hydrochloride (EDC, Sigma-Aldrich Chemical Co) was added to the mixture and reacted for 12 hr at 4° C. upon agitating. After the completion of reaction, the reaction mixture was filtered through a ultrafiltration cell (Millipore Co.) with a molecular weight cut off of 1 kDa, thereby yielding water soluble silicon-grafted poly(aspartic acid) with grafting ratio of ca. 26 mol %: ¹H NMR (300.40 MHz, D₂O): δ=4.3-4.21 (br, 1H), 3.35-3.12 (br, 2H), 2.82-2.70 (br, 2H), 1.04 (br, 2H), 0.66 (br, 2H).

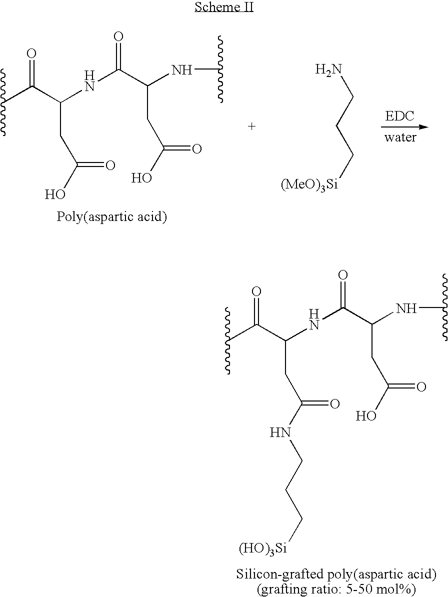

### EXAMPLE III

### Synthesis of Silicon-Grafted Chitosan

A water soluble silicon-grafted chitosan was prepared according to Scheme III.

1.6 g (10 mmole of —NH₂) of low molecular weight chitosan (5-20 kDa; KITTOLIFE Co., Seoul, South Korea) was dissolved in 50 mL distilled water. 0.99 g (4 mmol) of 3-(triethoxysilyl)propyl isocyanate (Sigma-Aldrich Chemical Co) in dimethylsulfoxide (DMSO) was added to the chitosan solution and reacted for 12 hr at room temperature upon agitating. After the completion of reaction, excess acetone (200 mL) was added to the resultant to collect precipitates, thereby yielding water soluble silicon-grafted chitosan with grafting ratio of ca. 31 mol %: ¹H NMR (300.40 MHz, D₂O): δ=5.38 (br, —O—CH—O), 5.25 (br, —O—CH—O), 4.0-3.15 (br), 2.92-2.7 (br), 1.31-1.13 (br, 2H), 0.68 (br, 2H).

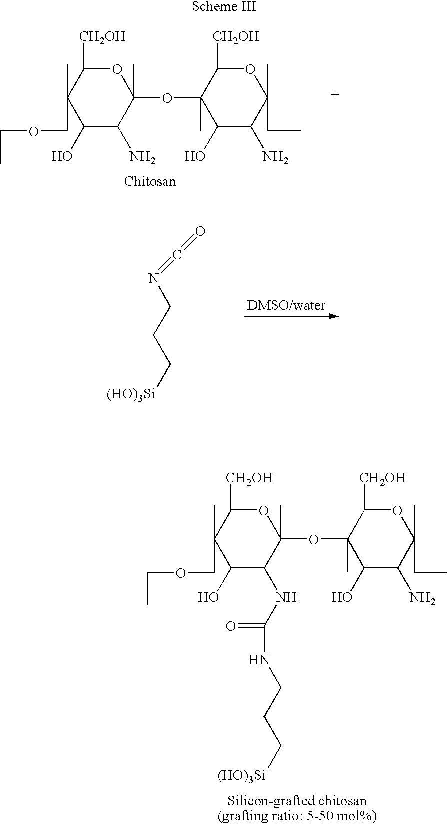

### EXAMPLE IV

### Synthesis of Silicon-Grafted Dextran

A water soluble silicon-grafted dextran was prepared according to Scheme IV.

1.0 of carboxymethyl dextran (CM-dextran sodium salt; Sigma-Aldrich, ca. 4 mmol of —COOH) and 0.72 g (4 mmol) of 3-aminopropyltrimethoxysilane (Sigma-Aldrich Chemical Co) were dissolved in 50 mL distilled water. 7.6 g (40 mmol) of 1-Ethyl-3-(3-dimethylaminopropyl) carbodiimide hydrochloride (EDC, Sigma-Aldrich Chemical Co) was added to the mixture and reacted for 12 hr at 4° C. upon agitating. After the completion of reaction, excess acetone (200 mL) was added to the resultant to collect precipitates, thereby yielding water soluble silicon-grafted dextran with grafting ratio of ca. 20 mol %: ¹H NMR (300.40 MHz, D₂O): δ=5.31 (br, —O—CH—O), 5.23 (br, —O—CH—O), 5.15-5.08 (br, 2H), 4.0-3.10 (br), 1.15 (br, 2H), 0.67 (br, 2H).

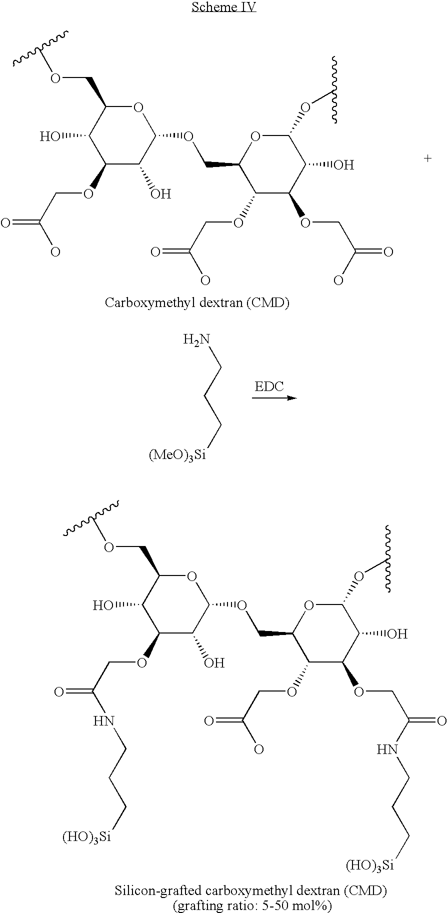

### EXAMPLE V

### Synthesis of Silicon-Grafted Heparin

A water soluble silicon-grafted heparin was prepared according to Scheme V.

0.6 g (1 mmole of —CO₂H) of heparin (sodium salt, Mw˜12 kDa, Cellsus Inc.) and 0.18 g (1 mmol) of 3-aminopropyltrimethoxysilane (Sigma-Aldrich Chemical Co) were dissolved in 20 mL distilled water. 1.9 g (10 mmol) of 1-Ethyl-3-(3-dimethylaminopropyl) carbodiimide hydrochloride (EDC, Sigma-Aldrich Chemical Co) was added to the mixture and reacted for 12 hr at 4° C. upon agitating. After the completion of reaction, excess acetone (100 mL) was added to the resultant to remove precipitates, thereby yielding water soluble silicon-grafted dextran with grafting ratio of ca. 28 mol %. ¹H NMR (300.40 MHz, D₂O): δ=5.4-5.2 (br, —O—CH—O), 4.20-4.05 (br), 4.0-2.95 (br), 1.20-1.10 (br, 2H), 0.65 (br, 2H).

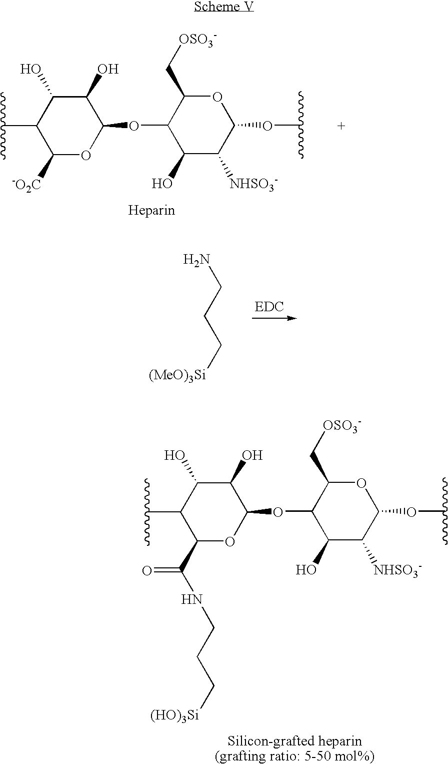

### EXAMPLE VI

### Preparation of Thermally Crosslinked (TCL)-SPION

FeCl₃.6H₂O (0.5 g, 1.85 mmol), and FeCl₂.4H₂O (0.184 g, 0.925 mmol) were dissolved in deoxygenated distilled water (30 mL) that had been degassed by bubbling with N₂ for 20 min. To this solution was added 7.5 mL of NH₄OH (˜28% in water) under N₂ atmosphere while stirring vigorously for 30 min. At this time, the pH of the mixture changed from approximately 2.3 to above 10.5, and the color changed to deep black, indicating the formation of iron oxide particles. To remove the remaining salt in the solution, an external magnetic field (Mₑₓₜ) was applied to the solution using a rare-earth magnet. Within a few minutes all of the black particles sank toward the magnet, and the resulting supernatant was discarded. Distilled water (30 mL) was added to the black precipitate, and the mixture was stirred gently to redisperse the particles. The particles were washed three times by applying Mₑₓₜ, followed by removal of the supernatant. After discarding the supernatant, 250 mg of the water soluble silicon-grafted polymers (e.g., hydrolyzed form of poly(TMSMA-r-PEGMA-r-NSA), silicon-grafted chitosan and silicon-grafted dextran) in 30 mL of distilled water was added, and the mixture was stirred for 1 hr. After several washing steps to remove the remaining polymer, the solution was sonicated in 30 mL of distilled water for 30 min at 200 W using a VCX-500 Ultrasonic Processor (Sonics & Materials, Inc., Newtown, Conn.). Next, Mₑₓₜ was applied again overnight to precipitate the aggregated particles. Most of the particles remained in the supernatant, but a small portion precipitated. The supernatant was carefully collected and then heated at 80° C. for 2 hr to achieve crosslinking between entangled polymer chains on the particle surface. The resulting thermally crosslinked polymer-coated SPION (TCL-SPION) were centrifuged at 6,000 rpm (3,580 g) for 10 min, followed by 10,000 rpm (9,950 g) for 10 min to further remove very small aggregates. The resulting TCL-SPION was stored at 4° C. until use in experiments.

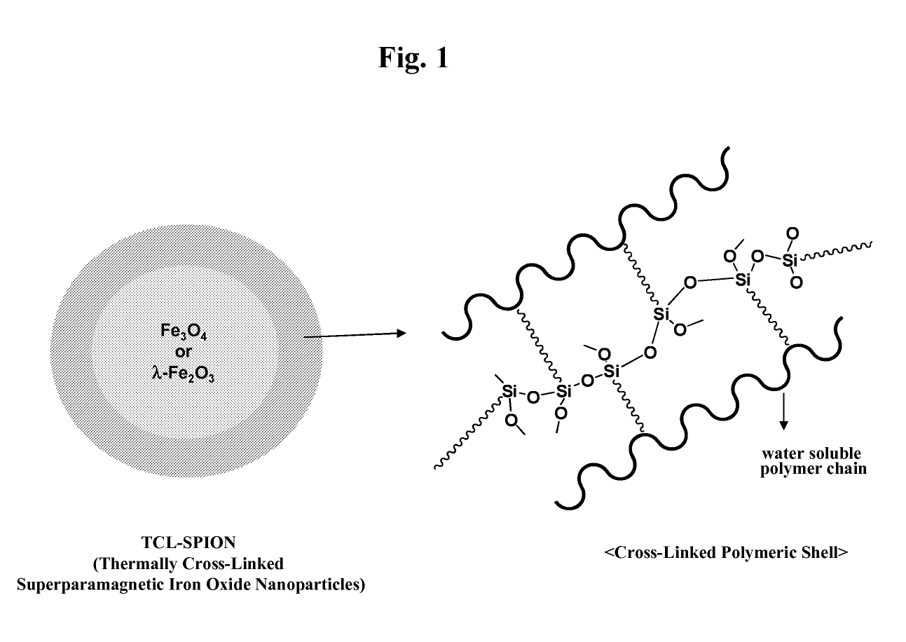

The TCL-SPION of this invention is schematically represented by FIG. 1. The polymeric shell coated on superparamagnetic iron oxide core is thermally crosslinked through hydroxylsilyl groups to form more compact and resistant polymer shell.

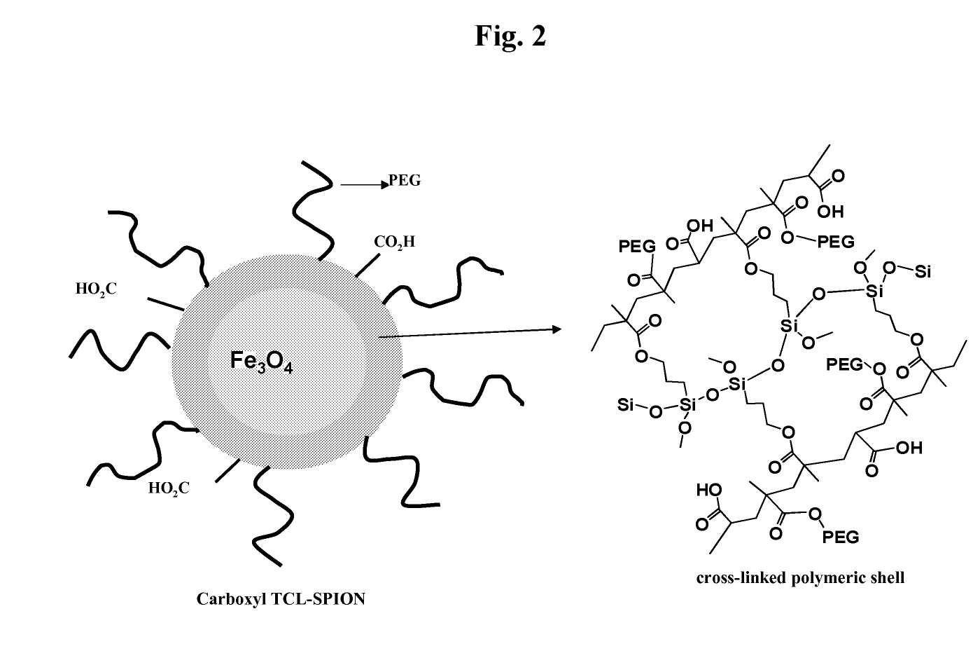

Meanwhile, where the hydrolyzed form (formula 1) of poly(TMSMA-r-PEGMA-r-NSA) was used, the carboxyl TCL-SPION was yielded as a final product. The thermally crosslinked carboxyl TCL-SPION of this invention is schematically represented by FIG. 2.

FT-IR spectroscopy (SPECTRUM 2000 of PERKIN ELMER) was used to confirm the presence of the polymers in the carboxyl TCL-SPION. The saturation magnetization (Ms) value of carboxyl TCL-SPION was measured by a magnetic property measurement system (MPMS) of Quantum Design at 300 K. The applied magnetic field was varied from 10 000 Oe to −10 000 Oe. Thermal gravimetric analysis (TGA) was carried out using TGA 2050 Thermogravimetric Analyzer (TA instruments). The temperature of the sample gradually increased from 40° C. to 600° C. at a rate of 10° C./min.

The functionalization of the carboxyl TCL-SPION can be changed to amino. The amino TCL-SPION was prepared as follows: 2,2′-(ethylenedioxy) bis-(ethylamine) (98%; Sigma-Aldrich Chemical Co, 0.2 mL of 1 M) and 1-ethyl-3-(3-dimethylaminopropyl) carbodiimide hydrochloride (1 mL of 100 mM, Sigma-Aldrich Chemical Co) were added successively to 1 mL of carboxyl TCL-SPION (24 mg SPION/mL) and vortexed vigorously. After 2.5 hr, the solution was dialyzed against distilled water using a 100-kDa molecular weight cutoff dialysis membrane (Spectrum Laboratories Inc., Rancho Dominguez, Calif.) for 2 days to synthesize amine-modified TCL-SPION (amino TCL-SPION). The synthesis of amino TCL-SPION was confirmed by measuring the surface zeta-potential change between carboxyl and amino TCL-SPION using the ELS 8000 electrophoretic light scattering apparatus. The resulting amino TCL-SPION was stored at 4° C. until use.

### EXAMPLE VII

### Preparation of Cy5.5 Conjugated TCL-SPION

Cy5.5 mono NHS ester (2 mg, Amersham Biosciences, France) dissolved in 400 μL of dimethyl sulfoxide was added slowly to 1 mL of amino TCL-SPION (14 mg SPION/mL) and reacted on ice in the dark with vigorous stirring. The Cy5.5 mono NHS ester used is represented by the formula 2.

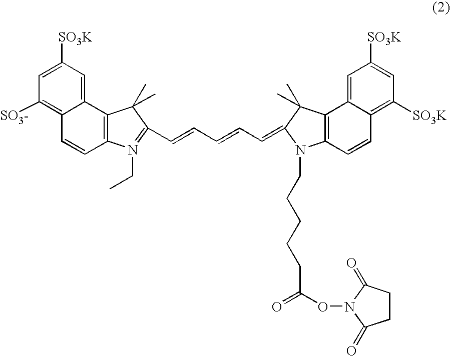

After 4 hr; unreacted free Cy5.5 was removed by gel filtration on Sephadex G-50 (Sigma-Aldrich Chemical Co). The resulting Cy5.5 TCL-SPION was stored at 4° C. in the dark until use. Quantitative analysis of the amount of conjugated Cy5.5 per milligram of SPION was determined by measuring the fluorescence intensity using an RF-5301PC spectrofluorometer (Shimadzu, Kyoto, Japan).

Fluorescence intensity of Cy5.5 conjugated TCL-SPION solution was measured in a 1.0 cm path length cuvette (Hellma Quartz Glass Cell, 10×2 mm) by RF-5301PC spectrofluorophotometer of SHIMADZU. The total area for the fluorescence intensity (λₑₓ=675 nm) was integrated from 685-740 nm with the excitation and emission slits set at 3 nm. The intensity was changed into μg (nmol) Cy5.5 per mg TCL-SPION by the Cy5.5/D.W. standard calibration graph.

### EXAMPLE VIII

### Cell Culture and Preparation

Lewis lung carcinoma (LLC) cells (American Type Culture Collection, Manassas, Va.) were grown as a monolayer in a humidified incubator in a 95% air/5% CO₂ atmosphere at 37° C. in Petri dish (Nunc, Naperville, Ill.) containing Dulbecco's Modified Eagle's Medium (GIBCO, Grand Island, N.Y.) supplemented with 10% (v/v) heat-inactivated fetal bovine serum (GIBCO), 100 IU/mL penicillin (GIBCO), and 100 IU/mL streptomycin (GIBCO). For experiments, LLC cells were detached mechanically and adjusted to the required concentration of viable cells as determined by counting in a hemocytometer.

### EXAMPLE IX

### In vivo MR and Optical Imaging

For all animals, MR and/or optical images were taken prior to and at selected time points after injection of TCL-SPION or Cy5.5 TCL-SPION. Mice were anesthetized by inhalation of 1.5% isoflurane in 1:2 O₂/N₂. The TCL-SPION was injected intravenously through the tail vein. MR imaging was performed with a 1.5-T MR scanner (GE Signa Excite Twin-speed; GE Health Care, Milwaukee, Wis.) and using an animal coil (4.3-cm Quadrature Volume Coil; Nova Medical System, Wilmington, Del., USA). For imaging of mice, T2-weighted fast-spin echo was performed under the following conditions: time of repetition/time of echo, 4200/102 ms; flip angle, 90°; echo train length, 10; field of view, 5 cm; section thickness, 2 mm; intersection gap, 0.2 mm; matrix, 256×160.

Quantitative analysis of MR images was performed by a single radiologist. The signal intensity (SI) was measured in defined regions of interest (ROIs), which were in similar locations within the tumor center. In addition, the SI of ROIs in the back muscle adjacent to the tumor was measured. The size of each ROI was two thirds the maximum diameter of the tumor. Relative signal enhancement was calculated by using SI measurements before (SI pre) and 3.5 h after (SI post) injection of TCL-SPION according to the following formula: relative signal enhancement (%)=100×[1−(SI post in tumor/SI post in muscle)/(SI pre in tumor/SI pre in muscle)], where SI pre: lesion signal intensity on pre-enhanced scan (control) and SI post: lesion signal intensity on post-enhanced scan at 3.5 hr.

Optical imaging was performed using an IVIS 100 imaging system (Xenogen Corp., Alameda, Calif.). The optical images were obtained 0 hr; 1 hr 45 min, and 3 hr 30 min after injection of Cy5.5 TCL-SPION. Optical images were acquired using an exposure time of 1 s and via the Cy5.5 filter channel.

### EXAMPLE X

### Measurements

The saturation magnetization (Ms) value of TCL-SPION was measured at 300 K using a magnetic property measurement system (Quantum Design, San Diego, Calif.). The applied magnetic field was varied from 10,000 to −10,000 Oe. The saturation magnetization in emu/g was normalized by the wt % of magnetite derived from thermogravimetric analysis (TGA) to obtain the emu/g iron. To confirm cross linking between entangled polymer chains on carboxyl TCL-SPION, X-ray photoelectron spectroscopy (XPS) was performed using a MultiLab 2000 (VG, East Grinstead, UK) with a non-monochromatic Mg Ka radiation X-ray source (300 watt), an incident angle of 65°, and an emission incident angle of 0°0. An analyzer pass energy of 20 eV was used to enable higher resolution (narrow) scans. The hydrodynamic particle sizes and zeta-potential of carboxyl, amino, and Cy5.5 TCL-SPION were measured using an ELS 8000 electrophoretic light scattering apparatus (Otsuka Electronics Korea, Seoul, South Korea). The size and dispersion quality of carboxyl and amino TCL-SPION were investigated by transmission electron microscopy (TEM) using a TECNAI F20 electron microscope (Philips Electronic Instruments Corp., Mahwah, N.J.) operated at 200 kV. For TEM sample preparation, carboxyl and amino TCL-SPION were diluted and deposited on a carbon-coated copper grid and allowed to air-dry.

### RESULTS AND DISCUSSION

To determine whether our strategy, the utilization of water soluble hydoxysilyl- or alkoxysilyl-functionalized polymeric shell thermally crosslinked for coating signal generating cores, is promising to provide excellent contrast agents for MR imaging and/or optical imaging, we synthesized a variety of TCL-SPIONs using a multitude of hydoxysilyl- or alkoxysilyl-functionalized polymers.

To introduce a functional group onto the surface of TCL-SPION, we designed the poly(TMSMA-r-PEGMA).³² PEG is needed for its anti-biofouling property, and silane part is needed to crosslink the polymer coating in TCL-SPION.³³ Thus, we simply added an additional functional group, a carboxylic acid in activated form with N-hydroxysuccinimide, to the poly(TMSMA-r-PEGMA) by incorporating the corresponding monomer during radical polymerization. ¹H NMR revealed that the resulting random copolymer, poly(PEGMA-r-TMSMA-r-NAS), contains a similar molar ratio of PEG, —Si(OCH₃)₃, and NHS-activated carboxylic acid (1:0.85:0.71) as the initial feed ratio (1:1:0.86) prior to polymerization. We chose the NHS-activated form of acrylic acid as a monomer because a polymeric gel was produced instead of the desired copolymer when the unprotected acrylic acid was used, presumably due to acid (COOH)-mediated crosslinking between partially hydrolyzed silane groups during radical polymerization at high temperature.³⁶

The poly(PEGMA-r-TMSMA-r-NAS) was hydrolyzed in water prior to use as a coating material of as-synthesized magnetite (Fe₃O₄) nanoparticles. During the hydrolysis process, trimethoxy silane and NHS ester are spontaneously converted to trihydroxy silane and carboxylic acid, respectively. The resulting polymer-coated SPION was prepared, which involves (i) generation of a magnetite core (Fe₃O₄), (ii) polymer coating, and (iii) purification.¹⁵ The polymer-coated SPION was dispersed in water and then heated at 80° C. for 2 hr to crosslink the polymer coating layer, resulting in TCL-SPION with a carboxyl group as a surface functional group. This preparation of carboxyl TCL-SPION is simpler and easier to carry out than the preparation of CLIO.

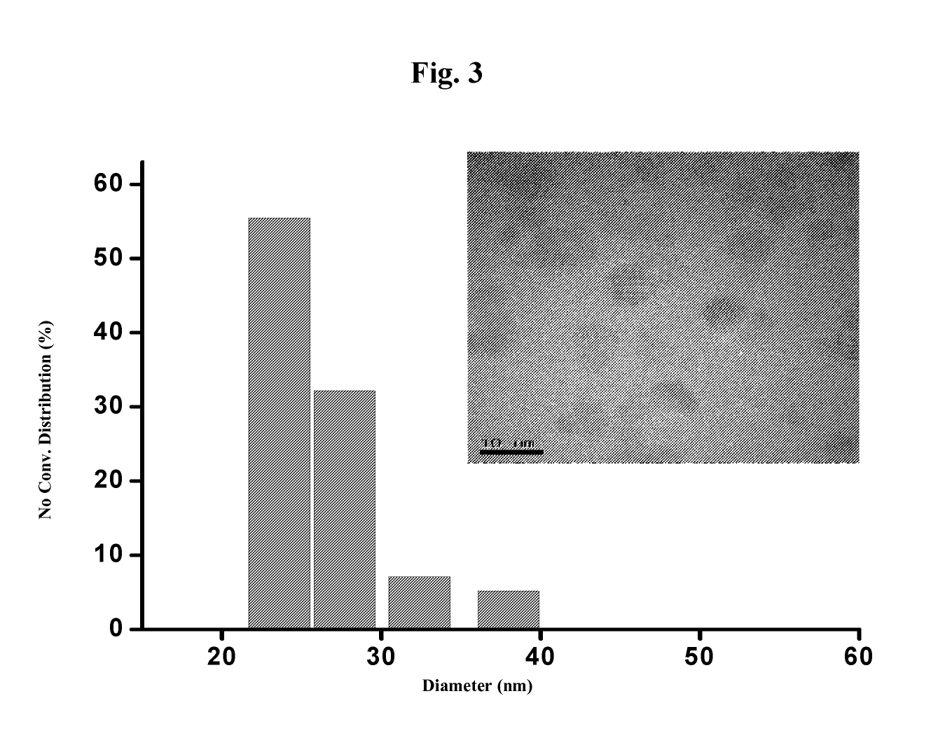

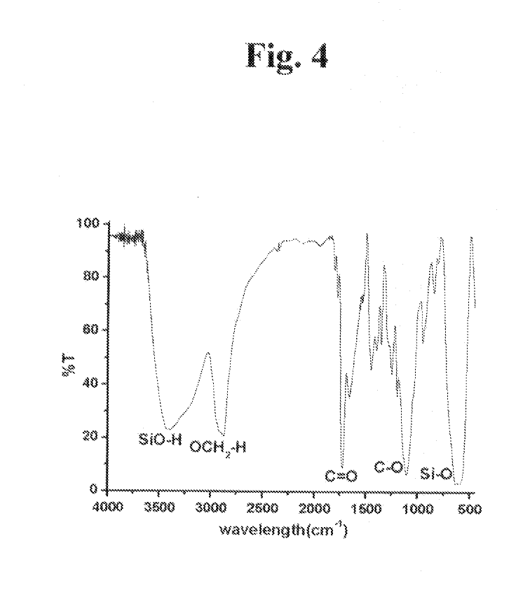

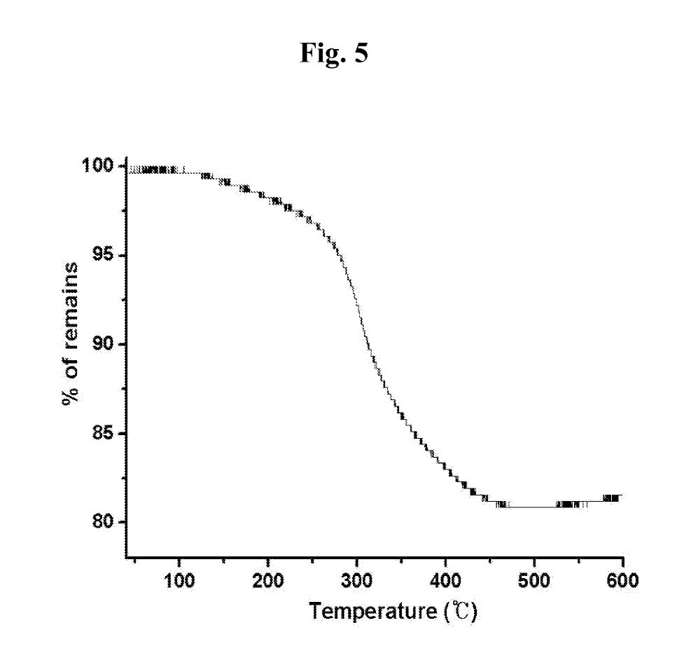

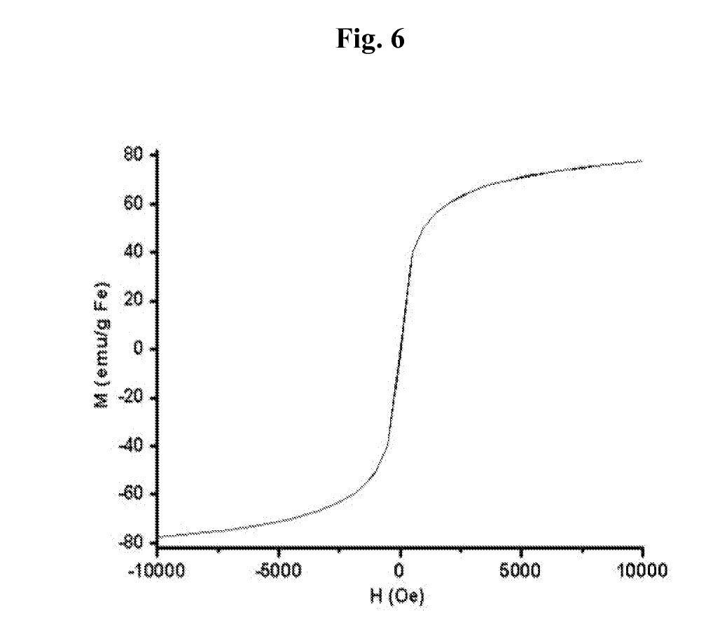

We next characterized the carboxyl TCL-SPION, including analysis of the particle size, surface charge, and strength of magnetization. Dynamic light scattering (DLS) measurements revealed that the carboxyl TCL-SPION had a relatively narrow size distribution with mean size of 25.6±2.7 nm (FIG. 3), which is similar to or smaller than conventional dextran-coated types of SPION such as CLIO^{23,27} and monocrystalline iron xide nanoparticle (MION)^{6,37}. Because DLS provides information on the hydrodynamic particle size of whole particle clusters, including polymer coating layers and the magnetite core, we examined the size of the iron oxide core alone by TEM. As shown in FIG. 2, the magnetite cores ranged from approximately 3 to 10 nm, indicating that the TCL-SPION were coated with a layer of polymer that was less than 10 nm thick. FT-IR spectroscopy of lyophilized carboxyl TCL-SPION confirmed the presence of the polymer in the nanoparticles, with characteristic peaks of hydrolyzed poly(TMSMA-r-PEGMA-r-NAS) around 1720, 1105, and 627 cm⁻¹, which correspond to stretching bands of C═O, C—O, and Si—O, respectively (FIG. 4). TGA further revealed that the polymer accounted for approximately 19% (w/w) of the carboxyl TCL-SPION (FIG. 5). Together, these results confirm the presence of a polymer coating on the SPION. When the magnetic moment was measured as a function of applied field at 300 K, the carboxyl TCL-SPION exhibited superparamagnetic behaviors showing high M_{S} of 77.5 emu/g Fe (FIG. 6). It is noteworthy that the M_{S} value for TCL-SPION in this work is much larger than other polymer-coated SPION which possess approximately 30-50 emu/g Fe.¹⁶

It is expected that —Si(OH)₃ groups of hydrolyzed poly(TMSMA-r-PEGMA-r-NAS) would be crosslinked upon heating, producing a highly stable polymer coating that should be advantageous for its in vivo use. To determine whether the crosslinking reaction occurred, we analyzed the surface of the polymer-coated nanoparticles by XPS before and after a 2 hr heat treatment at 80° C. High-resolution Si(2p) XPS of the polymer-coated SPION before heat treatment showed four different types of Si(2p) peaks at 97.88, 99.36, 100.75 and 102.13 eV (FIG. 3), which correspond to Si-metal (29.0%), Si(0) (17.8%), Si—OH (35.3%), and Si—O—C (17.9%), respectively. After heating, the peak corresponding to Si—OH significantly decreased from 35.3% to 28.9%, and a new peak characteristic of Si—O—Si bonds (12.5%) appeared at 101.46 eV, confirming crosslinking between Si—OH groups (Table 1).

TABLE 1

| | Composition (%) | | | | |
|---|---|---|---|---|---|
| Treatment | Si-metal | Si(0) | Si—OH | Si—O—C | Si—O—Si |
| Before heat | 29.0 | 17.8 | 35.3 | 17.9 | 0 |
| treatment | | | | | |
| After heat | 35.0 | 23.6 | 28.9 | 0 | 12.5 |
| treatment | | | | | |

Because the carboxyl TCL-SPION has carboxylic acids groups on its surface, additional functions such as near-infrared dyes for optical imaging and cancer-specific ligands for targeting can be introduced.³⁵ We prepared TCL-SPION conjugated with the near-infrared dye Cy5.5 to examine the feasibility of the nanoparticle system as a dual (MR/optical) imaging probe for cancer in vivo.^{35d,35g} Although a few reports have described MR/optical-based dual imaging of cancer using multifunctional nanoparticles in vitro,^{35a,35e} to our knowledge, only few of the dual imaging of cancer in vivo has been reported using such nanoparticles.^{35g}

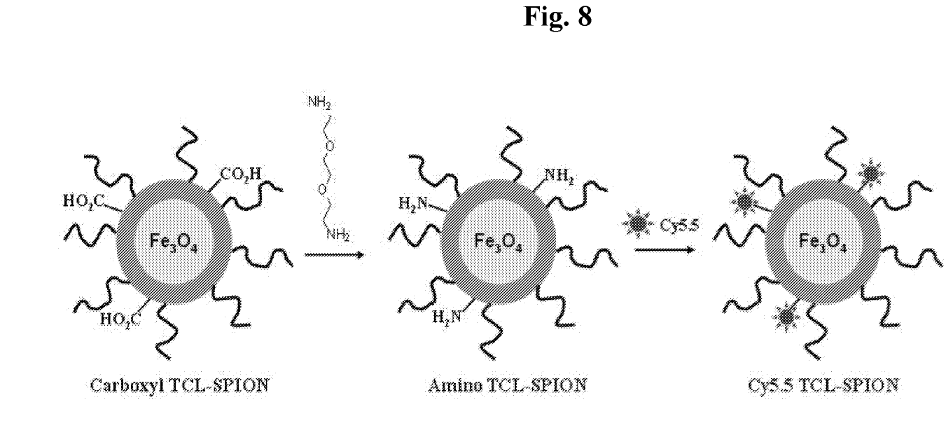

FIG. 8 depicts the synthetic scheme for the preparation of the Cy5.5-conjugated TCL-SPION. In the first step, the carboxyl TCL-SPION was converted to amino TCL-SPION after reaction with a hydrophilic bisamine reagent in aqueous solution. Next, NHS ester-activated Cy5.5 was reacted with the amino TCL-SPION to give covalently Cy5.5-conjugated TCL-SPION. We measured the particle sizes and zeta potentials of the nanoparticles at each step (Table 2). After amine modification, as expected, there was only a slight change in size (25.6±2.7 vs. 32.6±3.7 nm), but there was a remarkable increase in zeta potential from −30 to −1 mV, indicating that the negatively charged carboxylic acid surface was converted to a positively charged, amine-modified surface. Cy5.5 modification, however, led to a decrease in zeta potential due to the attachment of the highly negatively charged dye to the particle surface.

TABLE 2

| | Carboxyl | | Amino | | Cy5.5 | |
|---|---|---|---|---|---|---|
| Characteristics | TCL-SPION | | TCL-SPION | | TCL-SPION | |
| Size (DLS) | 25.6 ± 2.7 | nm | 32.6 ± 3.7 | nm | 31.9 ± 4.6 | nm |
| Zeta potential | −30.50 | mV | −1.34 | mV | −25.13 | mV |

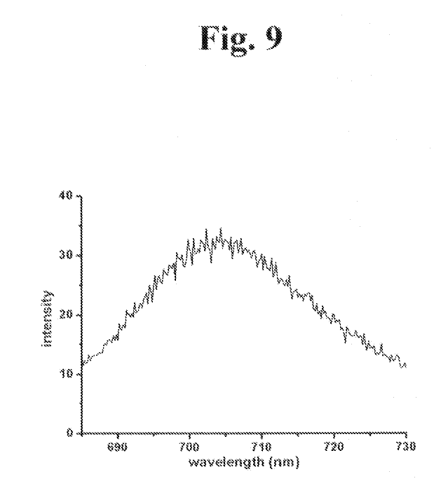

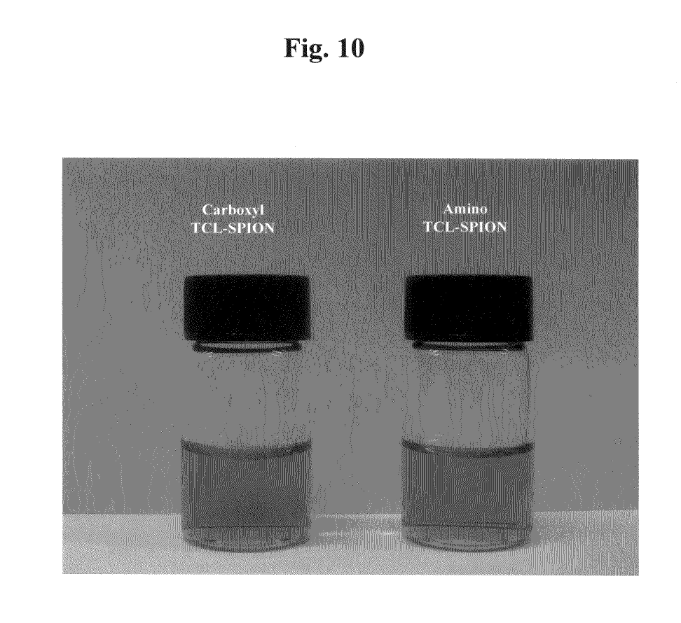

Measurement of the number of dye molecules per milligram of TCL-SPION revealed that approximately 0.02375 μg (0.021 nmol) of Cy5.5 dye was conjugated to each milligram of TCL-SPION (FIG. 9). On the other hand, both carboxyl and amino TCL-SPION dispersed well in water and were stable for up to a month under ambient conditions without any sign of aggregation (FIG. 10). This high stability may be attributed to the existence of a firm, crosslinked polymeric coating layer on SPION.

We next performed in vivo cancer imaging using the TCL-SPIONs of the present invention including the Cy5.5-conjugated TCL-SPION, silicon-grafted chitosan coated TCL-SPION and silicon-grafted carboxymethyl dextran coated TCL-SPION.

Crosslinked polymer coating layers of TCL-SPION provides high stability in physiological medium as well as resistance to uptake by cells in the reticuloendothelial system such as macrophages. We expected that TCL-SPIONs upon systemic circulation would be accumulated at tumor sites due to an enhanced permeability and retention effect as a result of leaky vasculature around tumors.³⁸ Tumor-bearing mice were prepared by subcutaneous injection of LLC cells into the flank above their upper left thighs. MR and optical fluorescence images of the tumor-bearing mice were taken in a sequential manner at selected time points before and after intravenous injection of Cy5.5 TCL-SPION (14.7 mg Fe/kg), silicon-grafted chitosan coated TCL-SPION 10 mg Fe/kg) or silicon-grafted carboxymethyl dextran coated TCL-SPION 10 mg Fe/kg) in phosphate-buffered saline.

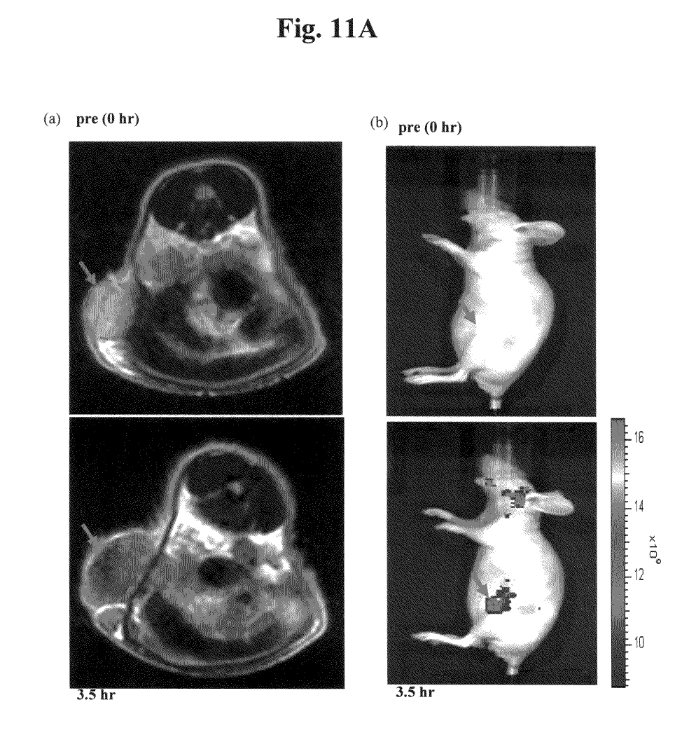

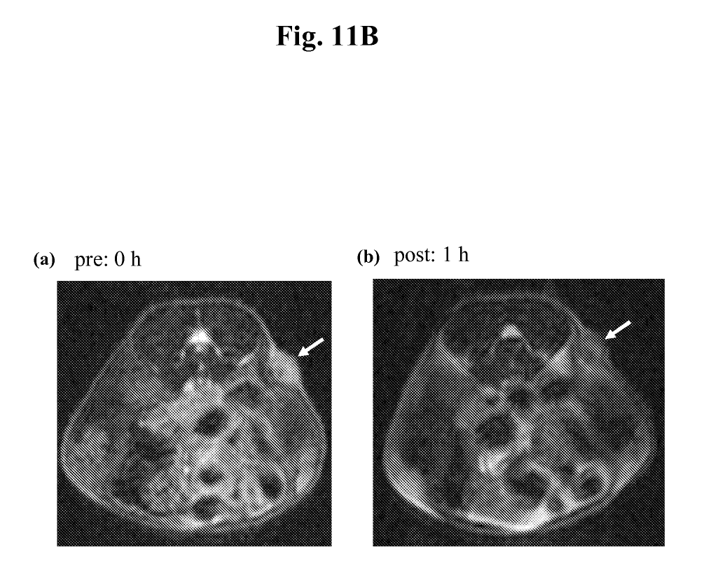

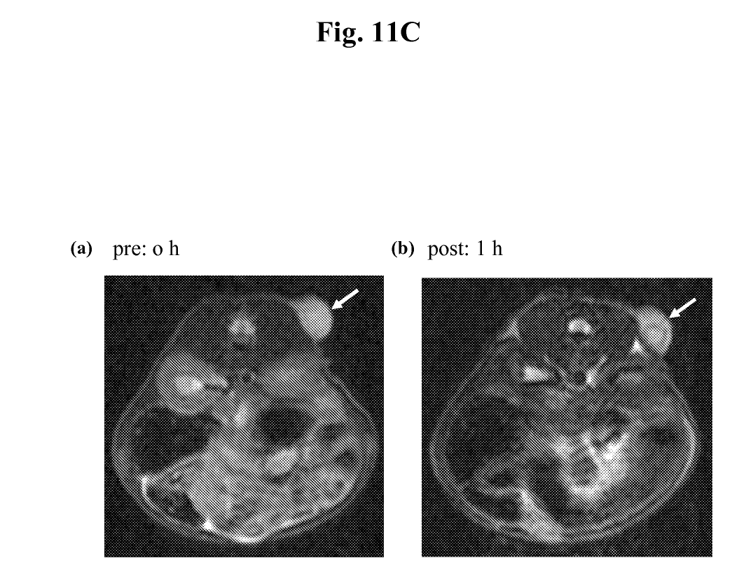

Before injection of TCL-SPIONs, the tumor appears as a hyperintense area in T2-weighted MR images (arrow and dashed circle in FIG. 11A, panel a, FIG. 11B, panel a and FIG. 11C, panel a). The relative signal intensity of the ROI in the T2-weighted image was calculated. At 3.5 hr post-injection of the Cy5.5 TCL-SPION, a noticeable darkening appeared in the tumor area in the T2-weighted MR image. The mean decrease in signal was 68% compared to pre-injection, indicating a large accumulation of the SPION within the tumor. This decrease in signal is sufficient for a radiologist to detect the tumor with high confidence.

In addition, at 1 hr post-injection of the silicon-grafted chitosan coated TCL-SPION, a noticeable darkening appeared in the tumor area in the T2-weighted MR image (FIG. 11B). The mean decrease in signal was 20% compared to pre-injection, indicating a large accumulation of the SPION even in much shorter period of time within the tumor. At 1 hr post-injection of the silicon-grafted carboxymethyl dextran coated TCL-SPION, a noticeable darkening appeared in the tumor area in the T2-weighted MR image (FIG. 11C). The mean decrease in signal was 28% compared to pre-injection, indicating a large accumulation of the SPION even in much shorter period of time within the tumor.

We also obtained in vivo fluorescence images of the same mouse at similar time points. At 3.5 hr post-injection, the pseudo color-adjusted optical images showed a relatively intense fluorescence signal exclusively in the tumor area (arrow in FIG. 11, panel b). In fact, accumulation of the Cy5.5 TCL-SPION in the tumor area was detectable even 1 hr post-injection (data not shown), indicating the rapid accumulation of TCL-SPION in the tumor. This finding agrees well with the MR imaging results. This appears to be the first successful use of a SPION system for MR/optical dual imaging of cancer in vivo. In addition, this imaging was carried out without attachment of specific targeting ligands to the nanoparticle surface.

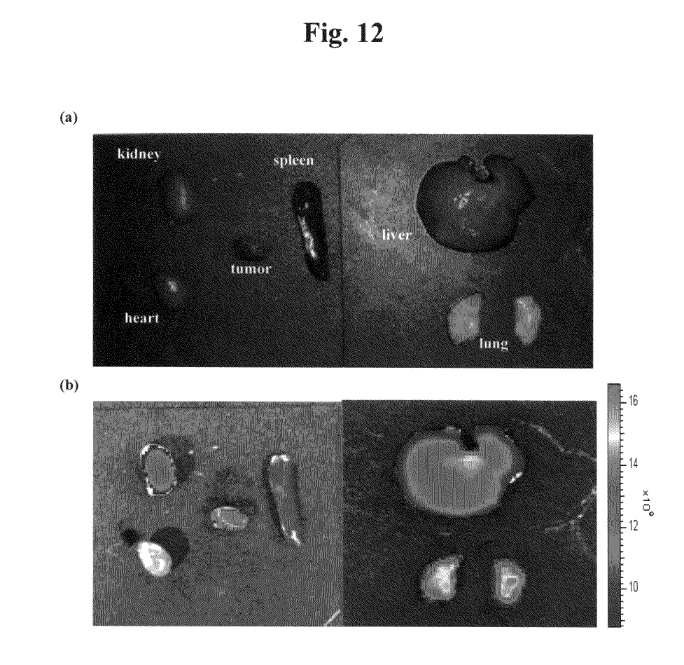

Because the signal intensity of fluorescence images in vivo depends on the penetration of light, deep-positioned tissues or organs may not be easily visualized by fluorescent methods.³⁹ We therefore harvested several major organs from the same mouse after MR/optical imaging and collected fluorescence images ex vivo to verify the accumulation of TCL-SPION in the tumor (FIG. 12). Consistent with the dual imaging results for whole mice, the highest fluorescence intensity was observed in the tumor. Although fluorescence was not detected in the heart and spleen, some fluorescence was observed in the kidney, liver, and lung. Because intravenously injected nanoparticles first arrive in the heart, followed by the lung, liver and then other tissues, we suspected that some aggregated nanoparticles are filtered by the lung and liver, resulting in the accumulation of a fluorescent signal. Despite this somewhat unfavorable biodistribution, the distinct difference in the relative accumulation between the tumor and other organs strongly suggests that these nanoparticles will be useful for dual imaging of cancer in vivo.

In conclusion, we fabricated a novel TCL-SPION containing functional groups, and demonstrated its potential use as a dual (MR/optical) imaging probe for cancer in viva Although Cy5.5-conjugated TCL-SPION does not possess any targeting ligands on its surface, it could efficiently detect tumors in vivo when analyzed by either MR or optical fluorescence imaging. Passive tumor targeting due to an enhanced permeability and retention effect could be achieved, presumably because of the antibiofouling and stable polymer coating layers on TCL-SPION. Because various functional molecules, such as targeting ligands and drugs, can be attached to the TCL-SPION, we are planning to develop multifunctional nanoparticles for cancer imaging and therapy as an extension of this work.

Having described a preferred embodiment of the present invention, it is to be understood that variants and modifications thereof falling within the spirit of the invention may become apparent to those skilled in this art, and the scope of this invention is to be determined by appended claims and their equivalents.

### REFERENCES

(1) (a) Farokhzad, O. C.; Langer, R. Adv. Drug Deliv. Rev. 2006, 58, 1456-1459. (b) Wagner, V.; Dullaart, A.; Bock, A. -K.; Zweck, A. Nat. Biotech. 2006, 24, 1211-1217. (c) Ferrari, M. Nat. Rev. Cancer 2005, 5, 161-171.
(2) Corot, C.; Robert, P.; Idee, J. M.; Port, M. Adv. Drug Deliv. Rev. 2006, 58, 1471-1504.
(3) Thorek, D. L.; Chen, A. K.; Czupryna, J.; Tsourkas, A. Ann. Biomed. Eng. 2006, 34, 23-38.
(4) Weissleder, R. Science 2006, 312, 1168-1171.
(5) (a) Lee, J. H.; Huh, Y. M.; Jun, Y. W.; Seo, J. W.; Jang, J. T; Song, H. T.; Kim, S.; Cho, E. J.; Yoon, H. G.; Suh, J. S.; Cheon, J., Artificially engineered magnetic nanoparticles for ultra-sensitive molecular imaging. Nat. Med. 2007, 13, 95-99. (b) Huh, Y. M.; Jun, Y. W.; Song, H. T.; Kim, S.; Choi, J. S.; Lee, J. H.; Yoon, S.; Kim, K. S.; Shin, J. S.; Suh, J. S.; Cheon, J. J. Am. Chem. Soc. 2005, 127, 12387-12391.
(6) Funovics, M. A.; Kapeller, B.; Hoeller, C.; Su, H. S.; Kunstfeld, R.; Puig, S.; Macfelda, K. Magn. Reson. Imaging 2004, 22, 843-850.
(7) Martina, M. S.; Fortin, J. P.; Menager, C.; Clement, O.; Barratt, G.; Grabielle-Madelmont, C.; Gazeau, F.; Cabuil, V.; Lesieur, S. J. Am. Chem. Soc. 2005, 127, 10676-10685.
(8) Weinmann, H. J.; Ebert, W.; Misselwitz, B.; Schmitt-Willich, H. Eur. J. Radiol. 2003, 46, 33-44.
(9) Harisinghani, M. G.; Barentsz, J.; Hahn, P. F.; Deserno, W. M.; Tabatabaei, S.; van de Kaa, C. H.; de la Rosette, J.; Weissleder, R. N. Engl. J. Med. 2003, 348, 2491-2499.
(10) de Vries, I. J.; Lesterhuis, W. J.; Barentsz, J. O.; Verdijk, P.; van Krieken, J. H.; Boerman, O. C.; Oyen, W. J.; Bonenkamp, J. J.; Boezeman, J. B.; Adema, G. J.; Bulte, J. W.; Scheenen, T. W.; Punt, C. J.; Heerschap, A.; Figdor, C. G. Nat. Biotechnol. 2005, 23, 1407-1413.
(11) Ito, A.; Kuga, Y.; Honda, H.; Kikkawa, H.; Horiuchi, A.; Watanabe, Y.; Kobayashi, T. Cancer Lett. 2004, 212, 167-175.
(12) Kohler, H.; Sun, C.; Wang, J.; Zhang, M. Langmuir 2005, 21, 8858-8864.
(13) Nasongkla, N.; Bey, E.; Ren, J.; Ai, H.; Khemtong, C.; Guthi, J. S.; Chin, S. F.; Sherry, A. D.; Boothman, D. A.; Gao, J., Nano Lett. 2006, 6, 2427-2430.
(14) Kim, J.; Park, S.; Lee, J. E.; Jin, S. M.; Lee, J. H.; Lee, I. S.; Yang, I.; Kim, J. S.; Kim, S. K.; Cho, M. H.; Hyeon, T. Angew. Chem. Int. Ed. 2006, 45, 7754-7758.
(15) Lee, H.; Lee, E.; Kim do, K.; Jang, N. K.; Jeong, Y. Y.; Jon, S. J. Am. Chem. Soc. 2006, 128, 7383-7389.
(16) Gupta, A. K.; Gupta, M. Biomaterials 2005, 26,3395-4021.
(17) Mikhaylova, M.; Kim, D. K.; Bobraysheva, N.; Osmolowsky, M.; Semenov, V.; Tsakalakos, T.; Muhammed, M. Langmuir 2004, 20, 2472-2477.
(18) Kohler, N.; Fryxell, G. E.; Zhang, M. J. Am. Chem. Soc. 2004, 126, 7206-7211.
(19) Gupta, A. K.; Curtis, A. S. J. Mater. Sci. Mater. Med. 2004, 15; 493-496.
(20) Raynal, I.; Prigent, P.; Peyramaure, S.; Najid, A.; Rebuzzi, C.; Corot, C. Invest. Radiol. 2004, 39, 56-63.
(21) Zhang, Y.; Kohler, N.; Zhang, M. Biomaterials 2002, 23, 1553-1561.
(22) Moghimi, S. M.; Hunter, A. C.; Murray, J. C. Pharmacol. Rev. 2001, 53, 283-318.
(23) Wang, Y.-X. J.; Hussain, S. M.; Krestin, G. P. Eur. Radiol. 2001, 11, 2319-2331.
(24) Berry, C. C.; Wells, S.; Charles, S.; Aitchison, G.; Curtis, A. S. Biomaterials 2004, 25, 5405-5413.
(25) Kim, D. K.; Mikhaylova, M.; Wang, F. H.; Kehr, J.; Bjelke, B.; Zhang, Y.; Tsakalakos, T.; Muhammed, M. Chem. Mat. 2003, 15, 4343-4351.
(26) D'Souza, A. J.; Schwen, R. L.; Topp, E. M. J. Control. Release 2004, 94, 91-100.
(27) Wunderbaldinger, P.; Josephson, L.; Weissleder, R. Acad. Radiol. 2002, Suppl 2, S304-306.
(28) Moore, A.; Marecos, E.; Bogdanov, A., Jr.; Weissleder, R. Radiology 2000, 214, 568-574.
(29) Josephson, L.; Tung, C, H.; Moore, A.; Weissleder, R. Bioconjugate Chem. 1999, 10, 186-191.
(30) Choi, H.; Choi, S. R.; Zhou, R.; Kung, H. F.; Chen, I. W. Acad. Radiol. 2004, 11, 996-1004.
(31) Tsourkas, A.; Shinde-Patil, V. R.; Kelly, K. A.; Patel, P.; Wolley, A.; Allport, J. R.; Weissleder, R. Bioconjugate Chem. 2005, 16, 576-581.
(32) Jon, S.; Seong, J.; Khademhosseini, A.; Tran, T. T.; Laibinis, P. E.; Langer, R. Langmuir 2003, 19, 9889-9893.
(33) (a) Khademhosseini, A.; Jon, S.; Suh, K. Y.; Tran, T -N. T; Eng, G.; Yeh, J.; Seong, J.; Langer, R. Adv. Mater. 2003, 15, 1995-2000. (b) Khademhosseini, A.; Suh, K. Y.; Jon. S.; Eng, G.; Yeh, J.; Chen. G. J.; Langer, R. Anal. Chem. 2004, 76, 3675-3681.
(34) (a) Josephson, L.; Kircher, M. F.; Mahmood, U.; Tang, Y.; Weissleder, R. Bioconjugated Chem. 2002, 13, 554-560. (b) Weissleder, R.; Kelly, K.; Sun, E. Y.; Shtatland, T.; Josephson, L. Nat. Biotechnol. 2005, 23, 1418-1423. (c) Lewin, M.; Carlesso, N.; Tung, C. H.; Tang, X. W.; Cory, D.; Scadden, D. T.; Weissleder, R. Nat. Biotechnol. 2000, 18, 410-414.
(35) (a) Veiseh, O.; Sun, C.; Gunn, J.; Kohler, N.; Gabikian, P.; Lee, D.; Bhattarai, N.; Ellenbogen, R.; Sze, R.; Hallahan, A.; Olson, 3.; Zhang, M. Nano Lett. 2005, 5, 1003-1008. (b) Talanov, V. S.; Regino, C. A.; Kobayashi, H.; Bernardo, M.; Choyke, P. L.; Brechbiel, M. W. Nano Lett. 2006, 6, 1459-1463. (c) Mulder, W. J.; Koole, R.; Brandwijk, R. J.; Storm, G.; Chin, P. T; Strijkers, G. J.; de Mello Donega, C.; Nicolay, K.; Griffioen, A. W. Nano Lett. 2006, 6, 1-6. (d) Orlova, A.; Magnusson, M.; Eriksson, T. L.; Nilsson, M.; Larsson, B.; Hoiden-Guthenberg, I.; Widstrom, C.; Carlsson, J.; Tolmachev, V.; Stahl, S.; Nilsson, F. Y., Tumor imaging using a picomolar affinity HER2 binding affibody molecule. Cancer Res. 2006, 66, 4339-4348. (e) Lee, J. H.; Jun, Y. W.; Yeon, S. I.; Shin, J. S.; Chen, J. Angew. Chem. Int. Ed. 2006, 45, 8160-8162. (f) Vuu, K.; Xie, J.; McDonald, M. A.; Bernardo, M.; Hunter, F.; Zhang, Y.; Li, K.; Bednarski, M.; Guccione, S. Bioconjugate Chem. 2005, 16, 995-999. (g) Kircher, M. F.; Mahmood, U.; King, R. S.; Weissleder, R.; Josephson, L. Cancer Res. 2003, 63, 8122-8125.
(36) Linssen, T.; Cassiers, K.; Cool, R; Vansant, E. F. Adv. Colloid Interface Sci. 2003, 103, 121-147.
(37) Weissleder, R.; Lee, A. S.; Khaw, B. A.; Shen, T.; Brady, T. J. Radiology 1992, 182, 381-385.
(38) (a) Maeda, H.; Matsumura, Y. Crit. Rev. Ther. Drug Carrier Syst. 1989, 6, 193-210. (b) Matsumura, Y.; Maeda, H. Cancer Res. 1986, 46, 6387-6392.
(39) (a) Troy, T; Jekic-McMullen, D.; Sambucetti, L.; Rice, B. Mol. Imaging 2004, 3, 9-23. (b) Weissleder, R. Nat. Biotech. 2001, 19, 316-317.

What is claimed is:

1. A contrast agent comprising a plurality of nanoparticles, wherein each of the nanoparticles comprises:
(a) a signal generating core; and
(b) a polymeric shell coated on the signal generating core, wherein the polymeric shell comprises a water soluble hydoxysilyl- or alkoxysilyl-functionalized polymer and wherein the polymer shell is thermally crosslinked through hydroxysilyl or alkoxysilyl groups of the polymer.

2. The contrast agent according to claim 1, wherein the signal generating core is a paramagnetic, superparamagnetic, proton density or X-ray active signal generating core.

3. The contrast agent according to claim 2, wherein the signal generating core is a superparamagnetic signal generating core containing an iron oxide.

4. The contrast agent according to claim 1, wherein the water soluble polymer comprises a synthetic polymer as main backbones.

5. The contrast agent according to claim 1, wherein the water soluble polymer comprises a natural polymer as main backbones.

6. The contrast agent according to claim 4, wherein the synthetic polymer as main backbones is selected from the groups consisting of poly(acrylic acid) and its derivates, poly(methacrylic acid) and its derivates, poly(acrylamide) and its derivates, poly(isocyanate) and its derivates, poly(styrene) and its derivates, poly(ethylene imine) and its derivates, poly(siloxane) and its derivates, poly(glutamic acid) and its derivates, poly(aspartic acid) and its derivates, poly(lysine) and its derivates, poly(arginine) and its derivates, polypropylene glycol, poly(vinyl alcohol) and its derivates, poly(vinyl pyrrolidone) and its derivates, and polyethylene oxide and its derivates.

7. The contrast agent according to claim 6, wherein the synthetic polymer as main backbones is poly(acrylic acid) or poly(methacrylic acid).

8. The contrast agent according to claim 5, wherein the natural polymer as main backbones is selected from the group consisting of chitosan and its derivatives, dextran and its derivatives, cellulose and its derivatives, heparin and its derivatives, and alginate and its derivatives.

9. The contrast agent according to claim 1, wherein the water soluble polymer is further functionalized with polyethylene glycol.

10. The contrast agent according to claim 3, wherein the thermally crosslinked polymeric shell formed by the silicon-containing water soluble polymer on the signal generating core has the relative silicon atomic composition of 5-50% in the form of Si—O—Si of all types of Si bonds in the polymeric shell.

11. The contrast agent according to claim 10, wherein the thermally crosslinked polymeric shell formed by the silicon-containing water soluble polymer on the signal generating core has the relative silicon atomic composition of 10-30% in the form of Si—O—Si of all types of Si bonds in the polymeric shell.

12. The contrast agent according to claim 10, wherein the thermally crosslinked polymeric shell formed by the silicon-containing water soluble polymer on the signal generating core has the relative silicon atomic composition of 10-30% in the form of Si—O—Si, 10-40% in the form of Si—OH, and 0-5% in the form of Si—O—C of all types of Si bonds in the polymeric shell.

13. The contrast agent according to claim 12, wherein the thermally crosslinked polymeric shell formed by the silicon-containing water soluble polymer on the signal generating core has the relative silicon atomic composition of 10-20% in the form of Si—O—Si, 18-34% in the form of Si—OH, and 0% in the form of Si—O—C of all types of Si bonds in the polymeric shell.

14. The contrast agent according to claim 1, wherein the polymeric shell further comprises an antibody, peptide, nucleic acid or dye.

15. The contrast agent according to claim 1, wherein the water soluble polymer prior to thermal crosslinking is represented by the following formula I: 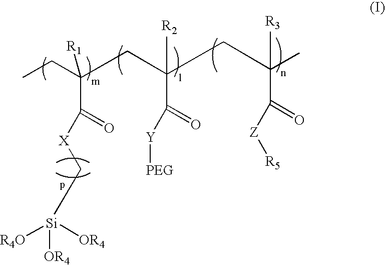
wherein R₁, R₂ and R₃ independently represent H or C₁-C₅ alkyl; R₄ represents H or C₁-C₅ alkyl; R₅ represents H, C₁-C₅ alkyl or heteroalkyl, (alkoxysilyl)alkyl, (hydroxysilyl)alkyl, 5-7 membered monocylic heterocycle consisting of carbon atoms and 1-3 heteroatoms, antibody, peptide, nucleic acid or dye; X, Y and Z independently represent oxygen or nitrogen atom; PEG represents polyethylene glycol; m, l and n independently represent an integer of 1-1,000; and p represents an integer of 1-10.

16. The contrast agent according to claim 1, wherein the water soluble polymer prior to thermal crosslinking comprises poly(aspartic acid) as main backbones represented by the following formula II: 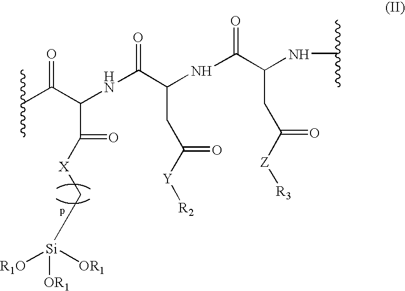
wherein R₁ represents H or C₁-C₅ alkyl; R₂ and R₃ independently represent H, C₁-C₅ alkyl or heteroalkyl, (alkoxysilyl)alkyl, (hydroxysilyl)alkyl, polyethylene glycol, antibody, peptide, nucleic acid or dye; X, Y and Z independently represent oxygen or nitrogen atom; and p represents an integer of 1-10.

17. The contrast agent according to claim 1, wherein the water soluble polymer prior to thermal crosslinking comprises chitosan as main backbones represented by the following formula III: 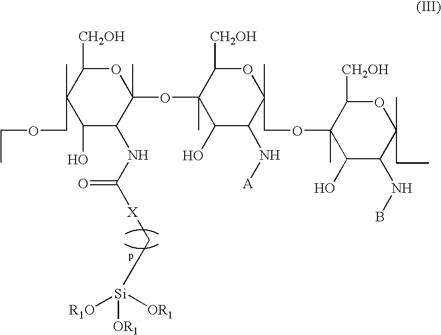
wherein R₁ represents H or C₁-C₅ alkyl; A and B independently represent H or —C(O)—Y—R₂; at least one of A and B is H; R₂ represents H, C₁-C₅ alkyl or heteroalkyl, (alkoxysilyl)alkyl, (hydroxysilyl)alkyl, polyethylene glycol, 5-7 membered monocylic heterocycle consisting of carbon atoms and 1-3 heteroatoms, antibody, peptide, nucleic acid or dye; X and Y independently represent oxygen or nitrogen atom; and p represents an integer of 1-10.

18. The contrast agent according to claim 1, wherein the water soluble polymer prior to thermal crosslinking comprises carboxymethyl dextran as main backbones represented by the following formula IV: 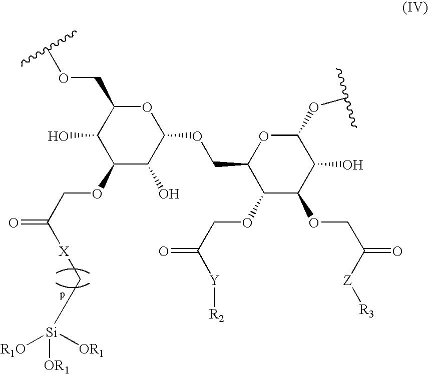
wherein R₁ represents H or C₁-C₅ alkyl; R₂ and R₃ independently represent H, C₁-C₅ alkyl or heteroalkyl, (alkoxysilyl)alkyl, (hydroxysilyl)alkyl, polyethylene glycol, antibody, peptide, nucleic acid or dye; X, Y and Z independently represent oxygen or nitrogen atom; and p represents an integer of 1-10.

19. The contrast agent according to claim 1, wherein the water soluble polymer prior to thermal crosslinking comprises heparin as main backbones represented by the following formula V: 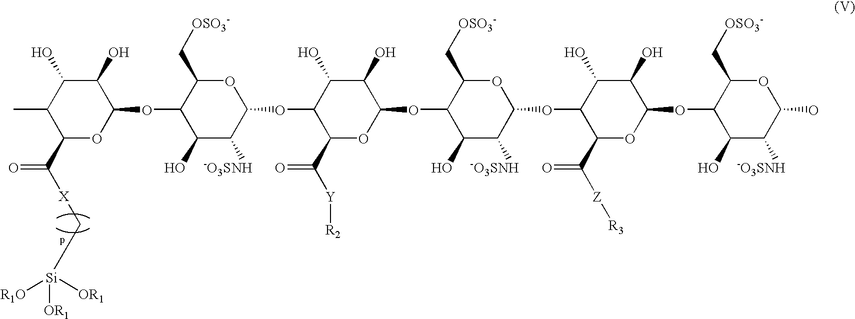
wherein R₁ represents H or C₁-C₅ alkyl; R₂ and R₃ independently represent H, C₁-C₅ alkyl or heteroalkyl, (alkoxysilyl)alkyl, (hydroxysilyl)alkyl, polyethylene glycol, antibody, peptide, nucleic acid or dye; X, Y and Z independently represent oxygen or nitrogen atom; and p represents an integer of 1-10.

20. The contrast agent according to claim 1, wherein the nanoparticle has a diameter in a range from 5 to 100 nm.

21. The contrast agent according to claim 1, wherein the nanoparticle has a saturation magnetization (Ms) value in a range from 10-200 emu/g Fe.

22. A process for preparing a contrast agent comprising a plurality of nanoparticles, which comprises the steps of:
(a) forming a superparamagnetic signal generating core containing iron oxide by use of salts of Fe²⁺ and Fe³⁺;
(b) forming a water soluble polymeric shell on the signal generating core by adding a water soluble hydoxysilyl- or alkoxysilyl-functionalized polymer to the signal generating core and agitating; and
(c) heating the resultant of step (b) to thermally crosslink the polymeric shell coated on the signal generating core, whereby the contrast agent comprising a plurality of nanoparticles is prepared.

23. The process according to claim 22, wherein the water soluble polymer comprises polyethylene glycol or is further functionalized with polyethylene glycol.

24. The process according to claim 22, wherein the thermally crosslinked polymeric shell on the signal generating core has the relative silicon atomic composition of 5-50% in the form of Si—O—Si of all types of Si bonds in the polymeric shell.

25. The process according to claim 24, wherein the thermally crosslinked polymeric shell on the signal generating core has the relative silicon atomic composition of 10-30% in the form of Si—O—Si of all types of Si bonds in the polymeric shell.

26. The process according to claim 25, wherein the thermally crosslinked polymeric shell on the signal generating core has the relative silicon atomic composition of 10-30% in the form of Si—O—Si, 10-40% in the form of Si—OH, and 0-5% in the form of Si—O—C of all types of Si bonds in the polymeric shell.

27. The process according to claim 26, wherein the thermally crosslinked polymeric shell on the signal generating core has the relative silicon atomic composition of 10-20% in the form of Si—O—Si, 18-34% in the form of Si—OH, and 0% in the form of Si—O—C of all types of Si bonds in the polymeric shell.

28. The process according to claim 22, wherein the polymeric shell further comprises an antibody, peptide, nucleic acid or dye.

29. The process according to claim 22, wherein the water soluble polymer prior to thermal crosslinking is represented by the following formula I: 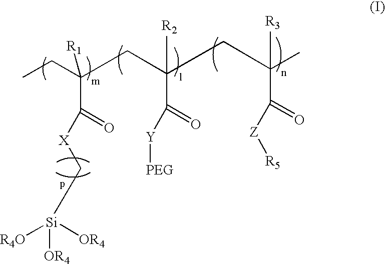
wherein R₁, R₂ and R₃ independently represent H or C₁-C₅ alkyl; R₄ represents H or C₁-C₅ alkyl; R₅ represents H, C₁-C₅ alkyl or heteroalkyl, (alkoxysilyl)alkyl, (hydroxysilyl)alkyl, 5-7 membered monocylic heterocycle consisting of carbon atoms and 1-3 heteroatoms, antibody, peptide, nucleic acid or dye; X, Y and Z independently represent oxygen or nitrogen atom; PEG represents polyethylene glycol; m, l and n independently represent an integer of 1-1,000; and p represents an integer of 1-10.

30. The process according to claim 22, wherein the water soluble polymer prior to thermal crosslinking comprises poly(aspartic acid) as main backbones represented by the following formula II: 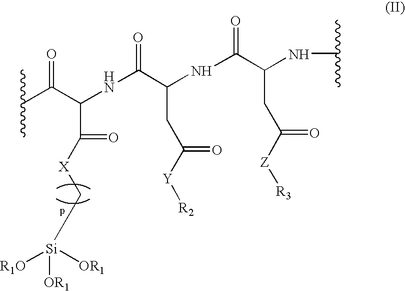
wherein R₁ represents H or C₁-C₅ alkyl; R₂ and R₃ independently represent H, C₁-C₅ alkyl or heteroalkyl, (alkoxysilyl)alkyl, (hydroxysilyl)alkyl, polyethylene glycol, antibody, peptide, nucleic acid or dye; X, Y and Z independently represent oxygen or nitrogen atom; and p represents an integer of 1-10.

31. The process according to claim 22, wherein the water soluble polymer prior to thermal crosslinking comprises chitosan as main backbones represented by the following formula III: 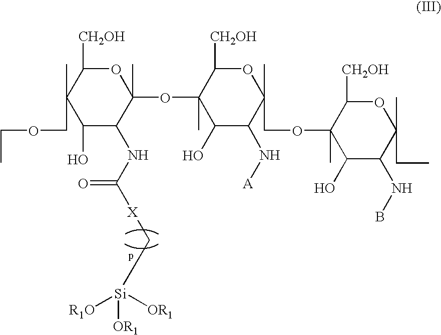
wherein R₁ represents H or C₁-C₅ alkyl; A and B independently represent H or —C(O)—Y—R₂; at least one of A and B is H; R₂ represents H, C₁-C₅ alkyl or heteroalkyl, (alkoxysilyl)alkyl, (hydroxysilyl)alkyl, polyethylene glycol, antibody, peptide, nucleic acid or dye; X and Y independently represent oxygen or nitrogen atom; and p represents an integer of 1-10.

32. The process according to claim 22, wherein the water soluble polymer prior to thermal crosslinking comprises carboxymethyl dextran as main backbones represented by the following formula IV: 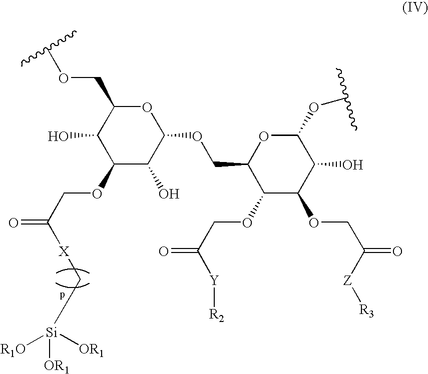
wherein R₁ represents H or C₁-C₅ alkyl; R₂ and R₃ independently represent H, C₁-C₅ alkyl or heteroalkyl, (alkoxysilyl)alkyl, (hydroxysilyl)alkyl, polyethylene glycol, antibody, peptide, nucleic acid or dye; X, Y and Z independently represent oxygen or nitrogen atom; and p represents an integer of 1-10.

33. The process according to claim 22, wherein the water soluble polymer prior to thermal crosslinking comprises heparin as main backbones represented by the following formula V: 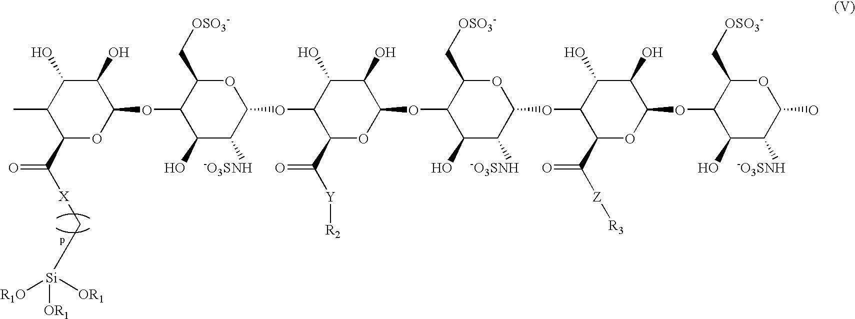
wherein R₁ represents H or C₁-C₅ alkyl; R₂ and R₃ independently represent H, C₁-C₅ alkyl or heteroalkyl, (alkoxysilyl)alkyl, (hydroxysilyl)alkyl, polyethylene glycol, antibody, peptide, nucleic acid or dye; X, Y and Z independently represent oxygen or nitrogen atom; and p represents an integer of 1-10.

34. A method for providing an image of an internal region of a patient, which comprises the steps of:
(a) administering to the patient a diagnostically effective amount of a contrast agent; wherein the contrast agent comprises a plurality of nanoparticles, wherein each of the nanoparticles comprises (i) a signal generating core containing an iron oxide; and (ii) a polymeric shell coated on the signal generating core, wherein the polymeric shell comprises a water soluble hydoxysilyl- or alkoxysilyl-functionalized polymer and wherein the polymer shell is thermally crosslinked through hydroxysilyl or alkoxysilyl groups of the polymer; and
(b) scanning the patient using a magnetic resonance imaging to obtain visible images of the region.

35. The method according to claim 34, wherein the water soluble polymer is further functionalized with polyethylene glycol.

36. The method according to claim 34, wherein the thermally crosslinked polymeric shell formed by the silicon-containing water soluble polymer on the signal generating core has the relative silicon atomic composition of 5-50% in the form of Si—O—Si of all types of Si bonds in the polymeric shell.

37. The method according to claim 36, wherein the thermally crosslinked polymeric shell formed by the silicon-containing water soluble polymer on the signal generating core has the relative silicon atomic composition of 10-30% in the form of Si—O—Si of all types of Si bonds in the polymeric shell.

38. The method according to claim 37, wherein the thermally crosslinked polymeric shell formed by the silicon-containing water soluble polymer on the signal generating core has the relative silicon atomic composition of 10-30% in the form of Si—O—Si, 10-40% in the form of Si—OH, and 0-5% in the form of Si—O—C of all types of Si bonds in the polymeric shell.

39. The method according to claim 38, wherein the thermally crosslinked polymeric shell formed by the silicon-containing water soluble polymer on the signal generating core has the relative silicon atomic composition of 10-20% in the form of Si—O—Si, 18-34% in the form of Si—OH, and 0% in the form of Si—O—C of all types of Si bonds in the polymeric shell.

40. The method according to claim 34, wherein the polymeric shell further comprises an antibody, peptide, nucleic acid or dye.

41. The method according to claim 34, wherein the water soluble polymer prior to thermal crosslinking is represented by the following formula I: 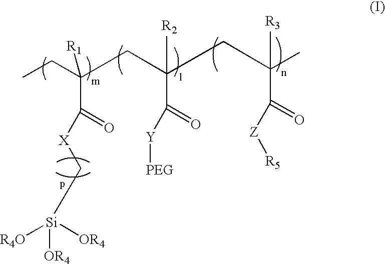
wherein R₁, R₂ and R₃ independently represent H or C₁-C₅ alkyl; R₄ represents H or C₁-C₅ alkyl; R₅ represents H, C₁-C₅ alkyl or heteroalkyl, (alkoxysilyl)alkyl, (hydroxysilyl)alkyl, 5-7 membered monocylic heterocycle consisting of carbon atoms and 1-3 heteroatoms, antibody, peptide, nucleic acid or dye; X, Y and Z independently represent oxygen or nitrogen atom; PEG represents polyethylene glycol; m, l and n independently represent an integer of 1-1,000; and p represents an integer of 1-10.

42. The method according to claim 34, wherein the water soluble polymer prior to thermal crosslinking comprises poly(aspartic acid) as main backbones represented by the following formula II: 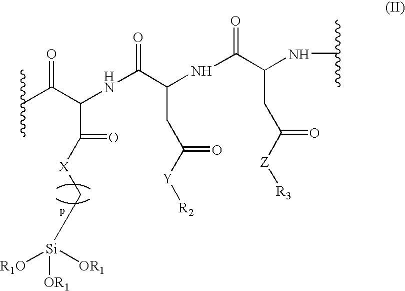
wherein R₁ represents H or C₁-C₅ alkyl; R₂ and R₃ independently represent H, C₁-C₅ alkyl or heteroalkyl, (alkoxysilyl)alkyl, (hydroxysilyl)alkyl, polyethylene glycol, antibody, peptide, nucleic acid or dye; X, Y and Z independently represent oxygen or nitrogen atom; and p represents an integer of 1-10.

43. The method according to claim 34, wherein the water soluble polymer prior to thermal crosslinking comprises chitosan as main backbones represented by the following formula III: 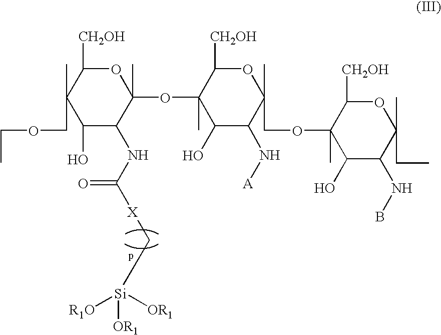
wherein R₁ represents H or C₁-C₅ alkyl; A and B independently represent H or —C(O)—Y—R₂; at least one of A and B is H; R₂ represents H, C₁-C₅ alkyl or heteroalkyl, (alkoxysilyl)alkyl, (hydroxysilyl)alkyl, polyethylene glycol, antibody, peptide, nucleic acid or dye; X and Y independently represent oxygen or nitrogen atom; and p represents an integer of 1-10.

44. The method according to claim 34, wherein the water soluble polymer prior to thermal crosslinking comprises carboxymethyl dextran as main backbones represented by the following formula IV: 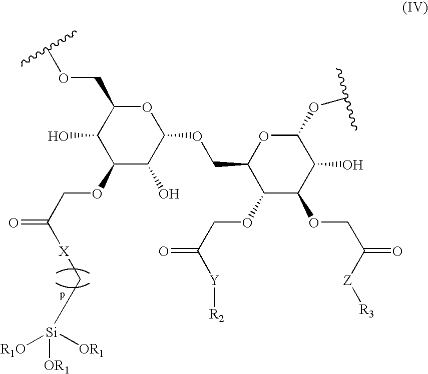
wherein R₁ represents H or C₁-C₅ alkyl; R₂ and R₃ independently represent H, C₁-C₅ alkyl or heteroalkyl, (alkoxysilyl)alkyl, (hydroxysilyl)alkyl, polyethylene glycol, antibody, peptide, nucleic acid or dye; X, Y and Z independently represent oxygen or nitrogen atom; and p represents an integer of 1-10.

45. The method according to claim 34, wherein the water soluble polymer prior to thermal crosslinking comprises heparin as main backbones represented by the following formula V: 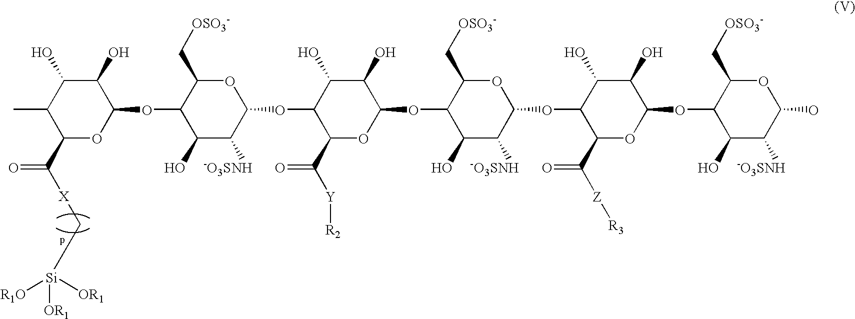
wherein R₁ represents H or C₁-C₅ alkyl; R₂ and R₃ independently represent H, C₁-C₅ alkyl or heteroalkyl, (alkoxysilyl)alkyl, (hydroxysilyl)alkyl, polyethylene glycol, antibody, peptide, nucleic acid or dye; X, Y and Z independently represent oxygen or nitrogen atom; and p represents an integer of 1-10.

46. The method according to claim 34, wherein the nanoparticle has a diameter in a range from 5 to 100 nm.

47. The method according to claim 34, wherein the nanoparticle has a saturation magnetization (Ms) value in a range from 10-200 emu/g Fe.
